(19) United States
(12) Patent Application Publication
(10) Pub. No.: US 2024/0226020 A1
(43) Pub. Date: Jul. 11, 2024

(54) SOTORASIB FORMULATION

(71) Applicant: AMGEN INC., Thousand Oaks, CA (US)

(72) Inventors: Fernando Antonio ALVAREZ-NUNEZ, Newbury Park, CA (US); Jiemin BAO, Camarillo, CA (US); Sai Prasanth CHAMARTHY, Oak Park, CA (US); Dominick Paul DAURIO, Camarillo, CA (US); Naga DUGGIRALA, Camarillo, CA (US); Brett E. HOUK, Newbury Park, CA (US); Yuan-Hon KIANG, Newbury Park, CA (US); Angela OLSOFSKY, Moorpark, CA (US); Namita SAWANT, Camarillo, CA (US)

(21) Appl. No.: 18/558,827

(22) PCT Filed: May 5, 2022
(86) PCT No.: PCT/US2022/027830

§ 371 Date: Nov. 3, 2023

Related U.S. Application Data
(60) Provisional application No. 63/184,941, filed on May 6, 2021.
(60) Provisional application No. 63/212,316, filed on Jun. 18, 2021.

(51) Int. Cl. A61K 9/28; A61K 9/00; A61K 9/20; A61K 31/519

(52) U.S. Cl. CPC A61K 9/284; A61K 9/0095; A61K 9/2009; A61K 9/2018; A61K 9/2054; A61K 9/2059; A61K 31/519

(57) ABSTRACT

The disclosure provides formulations comprising sotorasib (1), a diluent, a disintegrant and a lubricant.

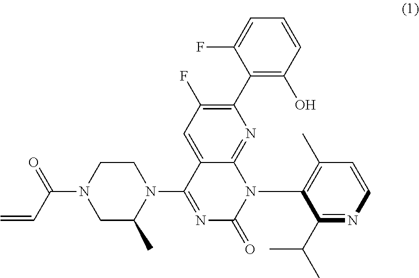

### CROSS-REFERENCE TO RELATED APPLICATIONS

This application claims the benefit of U.S. Provisional Patent Application No. 63/184,941, filed May 6, 2021, and U.S. Provisional Patent Application No. 63/212,316, filed Jun. 18, 2021, each of which is incorporated herein by reference in its entirety.

### BACKGROUND

From its identification as one of the first human oncogenes in 1982 (Der et al., 1982), KRAS (the Kirsten rat sarcoma viral oncogene homologue) has been the focus of extensive academic and industrial research, as a key node in the MAPK signal transduction pathway, as a transforming factor in a network of parallel effector pathways (e.g., PI3K/AKT) (Vojtek et al., 1998) and as a potential target for anti-cancer agents (Malumbres et al., 2003). Despite progress in the development of inhibitors of upstream and downstream nodes in the MAPK pathway (e.g., EGFR (Sridhar et al., 2003), BRAF (Holderfield et al., 2014), and MEK (Caunt et al., 2015), the KRAS protein has historically proven resistant to direct inhibition.

KRAS is a G-protein that couples extracellular mitogenic signaling to intracellular, pro-proliferative responses. KRAS serves as an intracellular “on/off” switch. Mitogen stimulation induces the binding of GTP to KRAS, bringing about a conformational change which enables the interaction of KRAS with downstream effector proteins, leading to cellular proliferation. Normally, pro-proliferative signaling is regulated by the action of GTPase-activating proteins (GAPs), which return KRAS to its GDP-bound, non-proliferative state. Mutations in KRAS impair the regulated cycling of KRAS between these GDP- and GTP-bound states, leading to the accumulation of the GTP-bound active state and dysregulated cellular proliferation (Simanshu et al., 2017).

Attempts to develop inhibitors of mutated KRAS proteins have historically been thwarted by the absence of druggable pockets on the surface of the protein (Cox et al., 2014). Discoveries in the field that followed brought about significant new efforts in KRAS inhibitor research, which have recently culminated in the entry of KRAS inhibitors into human clinical trials. See https://clinicaltrials.gov/: e.g., NCT03600883 & NCT04185883 (sotorasib, AMG 510) (last accessed Apr. 23, 2021). These efforts have recently culminated in the submission of a new drug application to the United States Food and Drug Administration for sotorasib (Amgen Press Release, Dec. 16, 2020; https://wwwext.amgen.com/newsroom/press-releases/2020/12/amgen-submits-sotorasib-new-drug-application-to-us-fda-for-advanced-or-metastatic-non-small-cell-lung-cancer-with-kras-g12c-mutation, last accessed Apr. 21, 2021).

Accordingly, there is a need for sotorasib formulations suitable for patients.

### SUMMARY

Provided herein are formulations of sotorasib. In one aspect, described herein are formulations comprising sotorasib, a diluent in an amount of 40-95% (w/w), a disintegrant in an amount of 0.5-5% (w/w), and a lubricant in an amount of 0.25-5% (w/w). In some embodiments, the formulations comprise sotorasib in an amount of 1-20% (w/w). In some embodiments, the formulations comprise sotorasib in an amount of 20-45% (w/w). In some embodiments, the formulations comprise the diluent in an amount of 61-91% (w/w). In some embodiments, the formulations comprise the diluent in an amount of 51-77% (w/w).

In another aspect, the formulations described herein are for use as a medicament, or for treating cancer.

In another aspect, described herein is a method of treating cancer in a patient comprising administering to the patient a therapeutically effective amount of sotorasib provided as a formulation described herein, wherein the formulation provides the therapeutically effective amount in one or more dosage units.

The terms “subject” and “patient” are used interchangeably herein. The terms “subjects” and “patients” are used interchangeably herein.

### BRIEF DESCRIPTION OF THE FIGURES

FIG. 1 is a graph showing the dissolution profile (percent dissolved over time) of sotorasib provided in Formulation #1 (1% (w/w), 1 mg sotorasib).

FIG. 2 is a graph showing the dissolution profile (percent dissolved over time) of sotorasib provided in Formulation #2 (37.5% (w/w), 240 mg sotorasib).

FIG. 3 is a graph showing the dissolution profile (percent dissolved over time) of sotorasib provided in Formulation #3 (50% (w/w), 360 mg sotorasib).

FIG. 4 is a graph showing the dissolution profile (percent dissolved over time) of sotorasib provided in Formulation #4 (30% (w/w), 180 mg sotorasib).

FIG. 5 is a graph showing the dissolution profile (percent dissolved over time) of sotorasib provided in Formulation #5 (40% (w/w), 360 mg sotorasib).

FIG. 6 is a graph showing the dissolution profile (percent dissolved over time) of sotorasib provided in Formulation #6 (20% (w/w), 30 mg sotorasib).

FIG. 7 is a graph showing the dissolution profile (percent dissolved over time) of sotorasib provided in Formulation #7 (20% (w/w), 120 mg sotorasib).

FIG. 8 is a graph showing the dissolution profile (percent dissolved over time) of sotorasib provided in Formulation #8 (20% (w/w), 120 mg sotorasib).

FIG. 9A is a graph showing the dissolution profile (percent dissolved over time) of sotorasib provided in Formulation #9a (32% (w/w), 240 mg sotorasib).

FIG. 9B is a graph showing the dissolution profile (percent dissolved over time) of sotorasib provided in Formulation #9b (32% (w/w), 240 mg sotorasib).

FIG. 10A is a graph showing the dissolution profile (percent dissolved over time) of sotorasib provided in Formulation #10a (32% (w/w), 320 mg sotorasib, Batch(a)).

FIG. 10B is a graph showing the dissolution profile (percent dissolved over time) of sotorasib provided in Formulation #10b (32% (w/w), 320 mg sotorasib, Batch (b)).

FIG. 11 is a graph showing the dissolution profiles for the following sotorasib formulations: (i) Formulation #8); (ii) Formulation #11; (iii) Formulation #12; (iv) Formulation #13.

FIG. 12A is a plot of tablet radial tensile strength (RTS) as a function of tablet solid fraction (also referred to as compactibility) for MCC lactose placebo blends.

FIG. 12B is a plot of tablet radial tensile strength (RTS) as a function of compaction pressure (also referred to as tabletability) for MCC lactose placebo blends.

FIG. 13A is a plot of tablet radial tensile strength (RTS) as a function of tablet solid fraction (SF) (also referred to as compactibility) for individual components including Avicel PH102, lactose 313 and sotorasib.

FIG. 13B is a plot of tablet radial tensile strength (RTS) as a function of compaction pressure (also referred to as tabletability) for individual components including Avicel PH102, lactose 313 and sotorasib.

FIG. 14A is a graph showing the flow energy profile of individual components including Avicel PH102, lactose 313 and sotorasib.

FIG. 14B is graph showing the change in volume (%) with respect to applied stress for individual components including Avicel PH102, lactose 313 and sotorasib.

### DETAILED DESCRIPTION

The present disclosure is based, in part, on the discovery that the formulations as disclosed herein comprising sotorasib and certain excipients in certain amounts result in immediate release formulations.

Further, the present disclosure is based, in part, on the discovery that the ratio of plastic to brittle excipients in a sotorasib formulation, e.g., in the form of a tablet, can affect the physical properties of such formulation. For example, as shown herein, a sotorasib formulation having a higher amount of plastic excipient (e.g., microcrystalline cellulose) and a lower amount of brittle excipient (e.g., lactose) was found to have issues with disintegration impacting the formulation's performance. In contrast, a sotorasib formulation, e.g., in the form of a tablet, having a lower amount of plastic excipient and a higher amount of brittle excipient was found to have poor tensile strength. Accordingly, a proper balance between overall brittleness and plasticity is required for a suitable formulation. As exemplified herein, provided are sotorasib tablet formulations comprising a ratio of plastic excipient to brittle excipient that does not have the above-noted tensile strength and tablet disintegration issues.

### Formulations

Sotorasib is a small molecule that specifically and irreversibly inhibits the KRAS G12C mutant protein. Sotorasib is also known as AMG 510 or 6-fluoro-7-(2-fluoro-6-hydroxyphenyl)-(1M)-1-[4-methyl-2-(propan-2-yl)pyridin-3-yl]-4-[(2S)-2-methyl-4-(prop-2-enoyl)piperazin-1-yl]pyrido[2,3-d]pyrimidin-2(1H)-one and has the following structure:

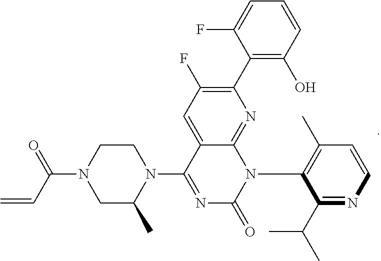

In one aspect, described herein is a formulation comprising sotorasib, a diluent in an amount of 50-95% (w/w), a disintegrant in an amount of 0.5-5% (w/w) and a lubricant in an amount of 0.25-5% (w/w). In another aspect, described herein is a formulation comprising sotorasib, a diluent in an amount of 40-95% (w/w), a disintegrant in an amount of 0.5-5% (w/w) and a lubricant in an amount of 0.25-5% (w/w).

In some embodiments, the formulations comprise sotorasib in an amount of 1% to about 50% (w/w) of In some embodiments, the formulations comprise sotorasib in an amount of 1-20% (w/w). In some embodiments, the formulations comprise sotorasib in an amount of 20-45% (w/w). In some embodiments, the formulations comprise sotorasib in an amount of 21-45% (w/w). In some embodiments, the formulations comprise sotorasib in an amount of 30-40% (w/w). In some embodiments, the formulations comprise sotorasib in an amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 21%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% (w/w) of the entire formulation.

In some embodiments, the formulations comprise sotorasib in an amount of 1 mg to about 400 mg. In some embodiments, the formulations comprise sotorasib in an amount of 1 mg to 360 mg, 30 mg to 120 mg, 180 mg to 320 mg, or 30 mg to 320 mg. In some embodiments, the formulations comprise sotorasib in an amount of about 1 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg. In some embodiments, the formulations comprise sotorasib in an amount of about 30 mg, or about 120 mg, or about 180 mg, or about 240 mg, or about 320 mg, or about 360 mg.

The formulations described herein comprise one or more diluents. Exemplary diluents include, but are not limited to, lactose, dibasic calcium phosphate (DCP), mannitol, sorbitol, xylitol, calcium carbonate, magnesium carbonate, tribasic calcium phosphate, trehalose, microcrystalline cellulose, and starch. In some embodiments, the diluent comprises one or more of lactose, dibasic calcium phosphate (DCP), mannitol, microcrystalline cellulose, and starch. In some embodiments, the diluent comprises one or more of lactose and microcrystalline cellulose. In some embodiments, the diluent comprises one or more of lactose and starch. In some embodiments, the diluent comprises one or more of lactose, dibasic calcium phosphate (DCP), and mannitol. In some embodiments, the starch is pregelatinized starch or corn starch. In some embodiments, the lactose is lactose monohydrate.

In some embodiments, the formulations comprise a diluent in an amount of 40% to about 95% (w/w). In some embodiments, the formulations comprise a diluent in an amount of 50% to about 95% (w/w). In some embodiments, the formulations comprise a diluent in an amount of 50% to about 90% (w/w). In some embodiments, the formulations comprise a diluent in an amount of about 61% to about 91% (w/w), or about 68% to about 84% (w/w), or about 51-77% (w/w), or 58-70% (w/w). In some embodiments, the formulations comprise a diluent in an amount of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, or about 77%, or about 78%, or about 79%, or about 80%, or about 81%, or about 82%, or about 83%, or about 84%, or about 85%, or about 86%, or about 87%, or about 88%, or about 89%, or about 90% (w/w).

Formulation components (e.g., a diluent) can, in general, be classified by the way in which they deform under compressive force, either by brittle fracture or by plastic deformation. The degree of deformation for a brittle material is independent of the rate and duration of the compression event (that is the compression applied), giving a strain rate sensitivity value for such materials of 0% (zero). Deformation of a plastic material is dependent on the rate and duration of the compression event and this is described by the strain rate sensitivity. When developing an tablet formulation, it is desirable to use a mixture of components: some with brittle character to minimize the strain rate sensitivity and some with moderate plastic character to increase the surfaces available to form bonds during compression. Excipients can be classified using the average Heckel yield pressure determined, for example, according to Zhang et al., 2017, which is herewith incorporated by reference in its entirety. An excipient having an average Heckel yield pressure greater than 125 MPa is considered a brittle excipient. An excipient having an average Heckel yield pressure less than 125 MPa is considered a plastic excipient. In some embodiments a plastic excipient has an average Heckel yield pressure of less than 100 MPa. In some embodiments a brittle excipient has an average Heckel yield pressure of more than 150 MPa. In some embodiments a plastic excipient has an average Heckel yield pressure of 50 MPa to 125 MPa. In some embodiments a brittle excipient has an average Heckel yield pressure of more than 125 MPa to 350 MPa.

In some embodiments, the formulations comprise a plastic diluent. Exemplary plastic diluents include, but are not limited to, microcrystalline cellulose and starch. In some embodiments, the starch is pregelatinized starch or corn starch.

In some embodiments, the formulations comprise a brittle diluent. Exemplary brittle diluents include, but are not limited to, lactose, dibasic calcium phosphate (DCP), mannitol, sorbitol, xylitol, calcium carbonate, magnesium carbonate, tribasic calcium phosphate, and trehalose. In some embodiments, the brittle diluent comprises one or more of lactose, dibasic calcium phosphate (DCP), or mannitol. In some embodiments, the brittle diluent is lactose. In some embodiments, the lactose is lactose monohydrate.

In some embodiments, the diluent comprises a plastic diluent and a brittle diluent, wherein the ratio by weight of the plastic diluent to the brittle diluent ranges from 2.5:1 to 3.5:1 (e.g., 2.5:1, 2.6:1, 2.7:1, 2.8:1, 2.9:1, 3:1, 3.1:1, 3.2:1, 3.3:1, 3.4:1 or 3.5:1). In some embodiments the diluent comprises a plastic diluent and a brittle diluent, wherein the ratio by weight of the plastic diluent to the brittle diluent ranges from 2.7:1 to 3.3:1. In some embodiments, the diluent comprises a plastic diluent and a brittle diluent, wherein the ratio by weight of the plastic diluent to the brittle diluent is 3:1.

In some embodiments, the diluent comprises a plastic diluent and optionally a brittle diluent, wherein the ratio by weight of the plastic diluent to sotorasib and the brittle diluent, if present, taken together, ranges from 1.2:1 to 1.7:1 (e.g., 1.2:1, 1.3:1, 1.4:1, 1.5:1, 1.6:1 or 1.7:1). In some embodiments, the diluent comprises a plastic diluent and optionally a brittle diluent, wherein the ratio by weight of the plastic diluent to sotorasib and the brittle diluent, if present, taken together, ranges from 1.4:1 to 1.5:1.

In some embodiments, the diluent comprises a plastic diluent and optionally a brittle diluent, and wherein (a) provided that the brittle diluent is present, the formulation is characterized by (1) a first ratio by weight of the plastic diluent to the brittle diluent that is greater than or equal to 2.5:1, 2.7:1, 3:1, 3.3:1, or 3.5:1; and (2) a second ratio by weight of the plastic diluent to sotorasib and the brittle diluent, taken together, is greater than or equal to 1.2:1, 1.4:1, 1.5:1, or 1.7:1 and less than the first ratio; or (b) provided that the brittle diluent is absent, the formulation is characterized by a ratio by weight of the plastic diluent to sotorasib that is greater than or equal to 1.2:1, 1.4:1, 1.5:1, or 1.7:1 and less than 2.5:1, 2.7:1, 3:1, 3.3:1, or 3.5:1. In some embodiments, the diluent comprises a plastic diluent and a brittle diluent, and wherein the first ratio is greater than or equal to 3:1 and the second ratio is greater than or equal to 1.4:1 and less than 3:1. In some embodiments, the diluent comprises a plastic diluent and no brittle diluent, and wherein the ratio by weight of the plastic diluent to sotorasib is greater than or equal to 1.4:1 and less than 3:1.

In some embodiments, the formulations comprise cellulose (e.g., microcrystalline cellulose) in the range of about 50% to about 75% (w/w) of the entire formulation, including any integer between the specified range. In some embodiments, the formulations comprise cellulose (e.g., microcrystalline cellulose) in the amount of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, or about 75% (w/w).

In some embodiments, the formulations comprise lactose (e.g., lactose monohydrate) in the range of about 19% to about 55% (w/w) of the entire formulation, including any integer between the specified range. In some embodiments, the formulations comprise lactose (e.g., lactose monohydrate) in the amount of about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, or about 55%.

In some embodiments, the formulations comprise 57% (w/w) microcrystalline cellulose and 19% (w/w) lactose monohydrate. In some embodiments, the formulations comprise 57% (w/w) microcrystalline cellulose and 7% (w/w) lactose monohydrate. In some embodiments, the formulations comprise 44% (w/w) microcrystalline cellulose and 14.5% (w/w) lactose monohydrate. In some embodiments, the formulations comprise 34.5% (w/w) microcrystalline cellulose and 11.5% (w/w) lactose monohydrate. In some embodiments, the formulations comprise 57% (w/w) microcrystalline cellulose and 9% (w/w) lactose monohydrate. In some embodiments, the formulations comprise 56% (w/w) microcrystalline cellulose. In some embodiments, the formulations do not comprise lactose.

In some embodiments, the weight percent ratio of microcrystalline cellulose to lactose monohydrate in the formulations is about 3:1 to about 1:1, including all iterations of ratios within the specified range. In other embodiments, the weight percent ratio of microcrystalline cellulose to lactose in the formulations is about 3:1.

### Disintegrant

The formulations described herein comprise a disintegrant. Exemplary disintegrants include, but are not limited to, cross-linked sodium carboxy methyl cellulose (croscarmellose sodium), cross-linked polyvinylpyrrolidone (crospovidone), sodium starch glycolate, pregelatinized starch, calcium carboxymethyl cellulose, low substituted hydroxypropyl cellulose, and magnesium aluminum silicate, and combinations thereof. In some embodiments, the disintegrant comprises one or more of croscarmellose sodium or sodium starch glycolate.

In some embodiments, the formulations comprise a disintegrant in an amount of about 0.5% to about 5% (w/w). In some embodiments, the formulation comprises a disintegrant in an amount of 3-5% (w/w) or 2-4% (w/w). In some embodiments, the amount of disintegrant in the formulations is about 0.5%, or about 0.6%, or about 0.7%, or about 0.8%, or about 0.9%, or about 1%, or about 2%, about 3%, or about 4%, or about 5% (w/w) of the entire formulation. In some embodiments, the formulations comprise a disintegrant in an amount of 3% (w/w). In some embodiments, the formulations comprise croscarmellose sodium in an amount of about 3% (w/w).

### Lubricant

The formulations described herein comprise a lubricant. Exemplary lubricants include, but are not limited to, magnesium stearate, calcium stearate, oleic acid, caprylic acid, stearic acid, magnesium isovalerate, calcium laurate, magnesium palmitate, behenic acid, glyceryl behenate, glyceryl stearate, sodium stearyl fumarate, potassium stearyl fumarate, zinc stearate, sodium oleate, sodium stearate, sodium benzoate, sodium acetate, sodium chloride, talc, polyethylene glycol, and hydrogenated vegetable oil. In some embodiments, the lubricant is magnesium stearate.

The amount of lubricant in the formulations is in the range of about 0.25% to about 5% (w/w) of the entire formulation. In some embodiments, the formulations comprise a disintegrant in an amount of 0.5-3% (w/w) or about 0.5-1.5% (w/w). In some embodiments, the amount of lubricant in the formulation is about 0.25%, about 0.3%, about 0.4%, about 0.5%, about 0.6%, about 0.7%, about 0.8%, about 0.9%, about 1%, about 2%, about 3%, about 4%, or about 5% (w/w) of the entire formulation.

In some embodiments, the formulations comprise sotorasib in an amount of 16-24% (w/w), a diluent in an amount of 61-91% (w/w), a disintegrant in an amount of 2.4-3.6% (w/w), and a lubricant in an amount of 0.8-1.2% (w/w). In some embodiments, the formulations comprise sotorasib in an amount of 18-22% (w/w) sotorasib, a diluent in an amount of 68-84% (w/w), a disintegrant in an amount of 2.7-3.3% (w/w), and a lubricant in an amount of 0.9-1.1% (w/w). In some embodiments, the formulations comprise sotorasib in an amount of 20% (w/w), a diluent in an amount of 76% (w/w), a disintegrant in an amount of 3% (w/w), and a lubricant in an amount of 1% (w/w). In some embodiments, the formulations comprise sotorasib in an amount of 30 mg. In some embodiments, the formulations comprise sotorasib in an amount of 120 mg.

In some embodiments, the formulations comprise sotorasib in an amount of 26-38% (w/w), a diluent in an amount of 51-77% (w/w), a disintegrant in an amount of 2.4-3.6% (w/w), and a lubricant in an amount of 0.8-1.2% (w/w). In some embodiments, the formulations comprise sotorasib in an amount of 29-35% (w/w) sotorasib, a diluent in an amount of 58-70% (w/w), a disintegrant in an amount of 2.7-3.3% (w/w), and a lubricant in an amount of 0.9-1.1% (w/w). In some embodiments, the formulations comprise sotorasib in an amount of 32% (w/w), a diluent in an amount of 64% (w/w), a disintegrant in an amount of 3% (w/w), and a lubricant in an amount of 1% (w/w). In some embodiments, the formulations comprise sotorasib in an amount of 240 mg. In some embodiments, the formulations comprise sotorasib in an amount of 320 mg.

### Coating Composition

In some embodiments, the formulation is coated with a coating composition. A coating composition may contain, for example, a membrane forming agent (e.g., a polymer), a plasticizer (which provides plasticity, flexibility, and extensibility to a coating membrane), a water-soluble base (e.g., lactose or sodium chloride), a dispersing agent (which prevents particles or tablets from adhering and aggregating after the coating). These components may be dissolved or dispersed in an appropriate solvent, such as water, alcohol, or the like, to prepare the coating composition.

Exemplary membrane forming agents include, for example, a water-insoluble polymer or a water-soluble polymer. The membrane forming agent is not particularly limited, so long as it is pharmaceutically acceptable and biocompatible. These membrane forming agents may be added alone or as a combination thereof in an appropriate amount(s).

Exemplary water-insoluble polymer include, but are not limited to, dibenzyl phthalate, dihexyl phthalate, butyl octyl phthalate, beeswax, carnauba wax, cetyl alcohol, cetyl stearyl alcohol, glyceryl behenate, lipids, fats, resins such as shellac or the like, cellulose derivatives such as ethyl cellulose, cellulose acetate, polyacrylate derivatives such as aminoalkylmethacryl copolymer (product name: Eudragit RS), polymethacrylate derivatives such as methacrylate copolymer (product name: Eudragit L), hydroxypropylmethyl cellulose acetate succinate, polylactic acid, and polyglycolic acid.

Exemplary water-soluble polymers include, but are not limited to, hypromellose, hydroxypropyl cellulose, hydroxyethyl cellulose, carmellose sodium, methyl cellulose, polyvinylpyrrolidone, polyethylene glycol, and polyvinyl alcohol.

In some embodiments, the coating composition comprises polyvinyl alcohol. In some embodiments, the coating composition further comprises one or more of titanium dioxide, polyethylene glycol, talc, and a coloring agent. Some exemplary coating compositions include ethylcellulose, polymethacrylates, as well as coating products sold by OPADRY™. In some embodiments, the coating agent is Opadry Clear, Opadry Blue 13B50579, Opadry White 33628707, Opadry QX 321A180025, or Opadry II (33G28707). In some embodiments the coating agent is Opadry White 33628707. In some embodiments the coating agent is Opadry QX 321A180025. In some embodiments the coating agent is Opadry II Yellow 85F120132. In some embodiments the coating agent is Opadry II Yellow 85F120222-CN. In some embodiments the coating agent is Opadry II Beige 85F170037.

In embodiments where the formulation is coated with a coating composition, the weight percentages of the excipients discussed throughout are with respect to the total weight of the formulation before the coating composition is applied.

### Preparation of Formulations

The formulations disclosed herein may be in any form suitable for oral administration, including, but not limited to, a tablet, a caplet, powder or granules encapsulated in capsules (e.g., soft or hard gelatin capsules), cachets or any sprinkle dosage form. In some embodiments, the formulations disclosed herein may be produced by dry granulation, wet granulation, melt extrusion, melt embedding or direct compression. In some embodiments, the formulations are produced by dry granulation or direct compression. In some embodiments, the formulations are produced by wet granulation. In some embodiments, the formulations are produced by dry granulation. In some embodiments, the formulations are produced by direct compression.

In some embodiments, the formulations are compressed to a tablet or a caplet. In accordance with these embodiments, the method of preparing the pharmaceutical composition may further comprise a step of compression. Suitable compression equipment includes, but is not limited to, mini press, single or double punch or rotary tablet press such as Killian, Korsch, Colton, Manesty, Stokes, Vector, and the like among others. Each possibility represents a separate embodiment. In some embodiments, the tablet or caplet is compressed using a compression force that affords a target hardness of about 40 N to about 150 N, including each integer within the specified range. Typical hardness values include, for example, about 50 N to about 130 N, preferably about 70 N to about 125 N, including each integer within the specified range. In certain embodiments, the tablet is further characterized by having friability of about 1% or less, for example about 0.2% to about 1%.

### Dissolution Profile

In some embodiments, at least 50% (e.g., at least 50%, at least 55%, at least 60%, at least 65%, at least 70%, at least 75%, at least 80%, or at least 85% or more) of the sotorasib in the formulation is released within 30 minutes as measured by a dissolution test using a USP <711> apparatus 2 with 75 rpm paddle speed, at 37° C. in a dissolution medium of 900 ml of water at pH 6.7 comprising 50 mM sodium phosphate and a surfactant to maintain sink conditions. In some embodiments, the surfactant is 0.2-0.5% (w/v) sodium dodecyl sulfate (SDS). In some embodiments, at least 80% of the sotorasib in the formulation is released within 30 minutes. In some embodiments, at least 85% of the sotorasib in the formulation is released within 15 minutes. In some embodiments, the formulations comprise sotorasib in an amount of 120 mg and the dissolution medium comprises 0.2% (w/v) sodium dodecyl sulfate (SDS). In some embodiments, the formulations comprise sotorasib in an amount of 240 mg and the dissolution medium comprises 0.5% (w/v) sodium dodecyl sulfate (SDS). In some embodiments, the formulations comprise sotorasib in an amount of 320 mg and the dissolution medium comprises 0.5% (w/v) sodium dodecyl sulfate (SDS).

### Methods of Treatment

Provided herein are methods of treating cancer in a patient, the method comprising administering to the patient a therapeutically effective amount of sotorasib provided in formulation described herein, wherein the formulation provides the therapeutically effective amount in one or more dosage units. In some embodiments, one or more cells of the cancer express a KRAS G12C mutant protein. In some embodiments, the therapeutically effect amount of sotorasib is 180 mg, 240 mg, 260 mg, 720 mg or 960 mg.

In some embodiments, the therapeutically effective amount is 240 mg. In some embodiments, the therapeutically effective amount is provided in two dosage units (e.g., 2×120 mg tablets).

In some embodiments, the therapeutically effective amount is provided by one dosage unit (e.g., 1×240 mg tablet).

In some embodiments, the therapeutically effective amount of sotorasib is 960 mg. In some embodiments, the therapeutically effective amount is provided in eight dosage units (e.g., 8×120 mg tablets). In some embodiments, the therapeutically effective amount is provided in four dosage units (e.g., 4×240 mg tablets). In some embodiments, the therapeutically effective amount is provided in three dosage units (e.g., 3×320 mg tablets).

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g., cancer. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality.

### KRAS G12C Cancers

Without wishing to be bound by any particular theory, the following is noted: sotorasib is a small molecule that specifically and irreversibly inhibits KRAS^{G12C} (Hong et al., 2020, at 1208). Hong et al. report that “[p]reclinical studies showed that [sotorasib] inhibited nearly all detectable phosphorylation of extracellular signal-regulated kinase (ERK), a key down-stream effector of KRAS, leading to durable complete tumor regression in mice bearing KRAS p.G12C tumors.” (id., see also Canon et al., 2019, and Lanman et al., 2020). Thus, in various embodiments, sotorasib at a total daily dose of 240 mg or 960 mg is disclosed for use in treating cancer, wherein one or more cells express KRAS G12C mutant protein.

Sotorasib was evaluated in a Phase 1 dose escalation and expansion trial with 129 subjects having histologically confirmed, locally advanced or metastatic cancer with the KRAS G12C mutation identified by local molecular testing on tumor tissues, including 59 subjects with non-small cell lung cancer, 42 subjects with colorectal cancer, and 28 subjects with other tumor types (Hong et al., 2020, at page 1208-1209). Hong et al. report a disease control rate (95% CI) of 88.1% for non-small cell lung cancer, 73.8% for colorectal cancer and 75.0% for other tumor types (Hong et al., 2020, at page 1213, Table 3). The cancer types showing either stable disease (SD) or partial response (PR) as reported by Hong et al. were non-small cell lung cancer, colorectal cancer, pancreatic cancer, appendiceal cancer, endometrial cancer, cancer of unknown primary, ampullary cancer, gastric cancer, small bowel cancer, sinonasal cancer, bile duct cancer, or melanoma (Hong et al., 2020, at page 1212 (Figure A), and Supplementary Appendix (page 59 (Figure S5) and page 63 (Figure S6)).

KRAS G12C mutations occur with the alteration frequencies shown in the table below (Cerami et al., 2012; Gao et al., 2013). For example, the table shows that 11.6% of subjects with non-small cell lung cancer have a cancer, wherein one or more cells express KRAS G12C mutant protein. Accordingly, sotorasib, which specifically and irreversibly bind to KRAS G12C is useful for treatment of subjects having a cancer, including, but not limited to the cancers listed in the table below.

TABLE

| | Alteration |
|---|---|
| Cancer Type | Frequency |
| Non-Small Cell Lung Cancer | 11.6 |
| Small Bowel Cancer | 4.2 |
| Appendiceal Cancer | 3.6 |
| Colorectal Cancer | 3.0 |
| Cancer of Unknown Primary | 2.9 |
| Endometrial Cancer | 1.3 |
| Mixed Cancer Types | 1.2 |
| Pancreatic Cancer | 1.0 |
| Hepatobiliary Cancer | 0.7 |
| Small Cell Lung Cancer | 0.7 |
| Cervical Cancer | 0.7 |
| Germ Cell Tumor | 0.6 |
| Ovarian Cancer | 0.5 |
| Gastrointestinal Neuroendocrine | 0.4 |
| Tumor | |
| Bladder Cancer | 0.4 |
| Myelodysplastic/Myeloproliferative | 0.3 |
| Neoplasms | |
| Head and Neck Cancer | 0.3 |
| Esophagogastric Cancer | 0.2 |
| Soft Tissue Sarcoma | 0.2 |
| Mesothelioma | 0.2 |
| Thyroid Cancer | 0.1 |
| Leukemia | 0.1 |
| Melanoma | 0.1 |

In various embodiments, the cancer is a solid tumor. In various embodiments, the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma. In some embodiments, the cancer is small bowel cancer, appendiceal cancer, endometrial cancer, hepatobiliary cancer, small cell lung cancer, cervical cancer, germ cell tumor, ovarian cancer, gastrointestinal neuroendocrine tumor, bladder cancer, myelodysplastic/myeloproliferative neoplasms, head and neck cancer, esophagogastric cancer, soft tissue sarcoma, mesothelioma, thyroid cancer, leukemia, or melanoma. In various embodiments, the cancer is non-small cell lung cancer, and in some specific embodiments, metastatic or locally advanced and unresectable non-small cell lung cancer. In various embodiments, the cancer is colorectal cancer. In some embodiments, the cancer is pancreatic cancer.

In some embodiments, the method further comprises dispersing the therapeutically effective amount provided as one or more dosage units in water by stirring before administration to the patient. In some embodiments, the water is non-carbonated. In some embodiments, the water has room temperature. In some embodiments, the water has a volume of 120 mL. In some embodiments, the therapeutically effective amount if dispersed in water immediately or within two hours before administration to the patient. In some embodiments, the patient has difficulty swallowing solids.

Methods of Detecting KRAS, STK11, KEAP1, EGFR, ALK, and/or ROS1 Mutation Status

The presence or absence of G12C, STK11, KEAP1, EGFR, ALK and/or ROS1 mutations in a cancer as described herein can be determined using methods known in the art. Determining whether a tumor or cancer comprises a mutation can be undertaken, for example, by assessing the nucleotide sequence encoding the protein, by assessing the amino acid sequence of the protein, or by assessing the characteristics of a putative mutant protein or any other suitable method known in the art. The nucleotide and amino acid sequences sequence of wild-type human KRAS (nucleotide sequence set forth in Genbank Accession No. BC010502; amino acid sequence set forth in Genbank Accession No. AG009594), STK11 (Gene ID: 6794; available at www.ncbi.nlm.nih.gov/gene/6794; accessed January 2020), KEAP1 (Gene ID: 9817; available at www.ncbi.nlm.nih.gov/gene/9817; accessed January 2020), EGFR (Gene ID: 1956; available at www.ncbi.nlm.nih.gov/gene/1956; accessed March 2021), ALK (Gene ID: 238; available at www.ncbi.nlm.nih.gov/gene/238; accessed March 2021), and ROS1 (Gene ID: 6098; available at www.ncbi.nlm.nih.gov/gene/6098; accessed March 2021) are known in the art.

Methods for detecting a mutation include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct and/or next generation-based sequencing, primer extension reactions, electrophoresis, oligonucleotide ligation assays, hybridization assays, TaqMan assays, SNP genotyping assays, high resolution melting assays and microarray analyses. In some embodiments, samples are evaluated for mutations, such as the KRAS G12C mutation, by real-time PCR. In real-time PCR, fluorescent probes specific for a certain mutation, such as the KRAS G12C mutation, are used. When a mutation is present, the probe binds and fluorescence is detected. In some embodiments, the mutation is identified using a direct sequencing method of specific regions in the gene. This technique identifies all possible mutations in the region sequenced. In some embodiments, gel electrophoresis, capillary electrophoresis, size exclusion chromatography, sequencing, and/or arrays can be used to detect the presence or absence of insertion mutations. In some embodiments, the methods include, but are not limited to, detection of a mutant using a binding agent (e.g., an antibody) specific for the mutant protein, protein electrophoresis and Western blotting, and direct peptide sequencing.

In some embodiments, multiplex PCR-based sequencing is used for mutation detection and can include a number of amplicons that provides improved sensitivity of detection of one or more genetic biomarkers. For example, multiplex PCR-based sequencing can include about 60 amplicons (e.g., 50, 51, 52, 53, 54, 55, 56, 57, 58, 59, 60, 61, 62, 63, 64, 65, 66, 67, 68, 69, or 70 amplicons). In some embodiments, multiplex PCR-based sequencing can include 61 amplicons. Amplicons produced using multiplex PCR-based sequencing can include nucleic acids having a length from about 15 bp to about 1000 bp (e.g., from about 25 bp to about 1000 bp, from about 35 bp to about 1000 bp, from about 50 bp to about 1000 bp, from about 100 bp to about 1000 bp, from about 250 bp to about 1000 bp, from about 500 bp to about 1000 bp, from about 750 bp to about 1000 bp, from about 15 bp to about 750 bp, from about 15 bp to about 500 bp, from about 15 bp to about 300 bp, from about 15 bp to about 200 bp, from about 15 bp to about 100 bp, from about 15 bp to about 80 bp, from about 15 bp to about 75 bp, from about 15 bp to about 50 bp, from about 15 bp to about 40 bp, from about 15 bp to about 30 bp, from about 15 bp to about 20 bp, from about 20 bp to about 100 bp, from about 25 bp to about 50 bp, or from about 30 bp to about 40 bp). For example, amplicons produced using multiplex PCR-based sequencing can include nucleic acids having a length of about 33 bp.

In some embodiments, the presence of one or more mutations present in a sample obtained from a patient is detected using sequencing technology (e.g., a next-generation sequencing technology). A variety of sequencing technologies are known in the art. For example, methods for detection and characterization of circulating tumor DNA in cell-free DNA can be described elsewhere (see, e.g., Haber and Velculescu, 2014). Non-limiting examples of such techniques include SafeSeqs (see, e.g., Kinde et al., 2011), OnTarget (see, e.g., Forshew et al., 2012), and TamSeq (see, e.g., Thompson et al., 2012).

In some embodiments, the presence of one or more mutations present in a sample obtained from a patient is detected using droplet digital PCR (ddPCR), a method that is known to be highly sensitive for mutation detection. In some embodiments, the presence of one or more mutations present in a sample obtained from a patient is detected using other sequencing technologies, including but not limited to, chain-termination techniques, shotgun techniques, sequencing-by-synthesis methods, methods that utilize microfluidics, other capture technologies, or any of the other sequencing techniques known in the art that are useful for detection of small amounts of DNA in a sample (e.g., ctDNA in a cell-free DNA sample).

In some embodiments, the presence of one or more mutations present in a sample obtained from a patient is detected using array-based methods. For example, the step of detecting a genetic alteration (e.g., one or more genetic alterations) in cell-free DNA is performed using a DNA microarray. In some embodiments, a DNA microarray can detect one more of a plurality of cancer cell mutations. In some embodiments, cell-free DNA is amplified prior to detecting the genetic alteration. Non-limiting examples of array-based methods that can be used in any of the methods described herein, include: a complementary DNA (cDNA) microarray (see, e.g., Kumar et al. 2012; Laere et al. 2009; Mackay et al. 2003; Alizadeh et al. 1996), an oligonucleotide microarray (see, e.g., Kim et al. 2006; Lodes et al. 2009), a bacterial artificial chromosome (BAC) clone chip (see, e.g., Chung et al. 2004; Thomas et al. 2005), a single-nucleotide polymorphism (SNP) microarray (see, e.g., Mao et al. 2007; Jasmine et al. 2012), a microarray-based comparative genomic hybridization array (array-CGH) (see, e.g., Beers and Nederlof, 2006; Pinkel et al. 2005; Michels et al. 2007), a molecular inversion probe (MIP) assay (see, e.g., Wang et al. 2012; Lin et al. 2010). In some embodiments, the cDNA microarray is an Affymetrix microarray (see, e.g., Irizarry 2003; Dalma-Weiszhausz et al. 2006), a NimbleGen microarray (see, e.g., Wei et al. 2008; Albert et al. 2007), an Agilent microarray (see, e.g., Hughes et al. 2001), or a BeadArray array (see, e.g., Liu et al. 2017). In some embodiments, the oligonucleotide microarray is a DNA tiling array (see, e.g., Mockler and Ecker, 2005; Bertone et al. 2006). Other suitable array-based methods are known in the art. Methods for determining whether a tumor or cancer comprises a mutation can use a variety of samples. In some embodiments, the sample is taken from a patient having a tumor or cancer. In some embodiments, the sample is a fresh tumor/cancer sample. In some embodiments, the sample is a frozen tumor/cancer sample. In some embodiments, the sample is a formalin-fixed paraffin-embedded (FFPE) sample. In some embodiments, the sample is a circulating cell-free DNA and/or circulating tumor cell (CTC) sample. In some embodiments, the sample is processed to a cell lysate. In some embodiments, the sample is processed to DNA or RNA. In a certain embodiment, the sample is acquired by resection, core needle biopsy (CNB), fine needle aspiration (FNA), collection of urine, or collection of hair follicles. In some embodiments, a liquid biopsy test using whole blood or cerebral spinal fluid may be used to assess mutation status.

In various embodiments, a test approved by a regulatory authority, such as the US Food and Drug Administration (FDA), is used to determine whether the patient has a mutation, e.g., a KRAS G12C mutated cancer, or whether the tumor or tissue sample obtained from such patient contains cells with a mutation. In some embodiments, the test for a KRAS mutation used is Therascreen® KRAS RGQ PCR Kit (Qiagen). The Therascreen® KRAS RGQ PCR Kit is a real-time qualitative PCR assay for the detection of 7 somatic mutations in codons 12 and 13 of the human KRAS oncogene (G12A, G12D, G12R, G12C, G12S, G12V, and G13D) using the Rotor-Gene Q MDx 5plex HRM instrument. The kit is intended for use with DNA extracted from FFPE samples of NSCLC samples acquired by resection, CNB, or FNA. Mutation testing for STK11, KEAP1, EGFR, ALK and/or ROS1 can be conducted with commercially available tests, such as the Resolution Bioscience Resolution ctDx Lung™ assay that includes 24 genes (including those actionable in NSCLC). Tissue samples may be tested using Tempus xT 648 panel.

In some embodiments, the cancer has been identified as having a KRAS G12C mutation. In some embodiments, the cancer has been identified as having a mutation of STK11, e.g., a loss-of-function mutation. In some embodiments, the cancer has been identified as having a mutation of KEAP1, e.g., a loss-of-function mutation. In some embodiments, the cancer has been identified as having wild-type STK11. In some embodiments, the cancer has been identified as having wild-type KEAP1.

In various embodiments, the cancer has been identified as having a loss-of-function mutation of STK11 and wild-type KEAP1. In some embodiments, the cancer has been identified as having a loss-of-function mutation of STK11 and a loss-of-function mutation of KEAP1. In some embodiments, the cancer has been identified as having wild-type of STK11 and wild-type KEAP1. In some embodiments, the cancer has been identified as having wild-type of STK11 and a loss-of-function mutation of KEAP1.

The term “loss-of-function mutation” as used herein refers to a mutation (e.g., a substitution, deletion, truncation, or frameshift mutation) that results in expression of a mutant protein that no longer exhibits wild-type activity (e.g., reduced or eliminated wild-type biological activity or enzymatic activity), results in expression of only a fragment of the protein that no longer exhibits wild-type activity, or results in no expression of the wild-type protein. For example, a loss-of-function mutation affecting the STK11 gene in a cell may result in the loss of expression of the STK11 protein, expression of only a fragment of the STK11 protein, or expression of the STK11 protein that exhibits diminished or no enzymatic activity (e.g., no serine/threonine kinase enzymatic activity) in the cancerous cell. Similarly, a loss-of-function mutation affecting the KEAP1 gene in a cell may result in the loss of expression of the KEAP1 protein, expression of only a fragment of the KEAP1 protein, or expression of a KEAP1 protein that exhibits diminished or no activity (e.g., inability to interact with or activate Nuclear factor erythroid 2-related factor 2 (NRF2)) in the cell.

### Methods of Detecting PD-L1 Protein Expression

PD-L1 expression can be determined by methods known in the art. For example, PD-L1 expression can be detected using PD-L1 IHC 22C3 pharmDx, an FDA-approved in vitro diagnostic immunohistochemistry (IHC) test developed by Dako and Bristol-Meyers Squibb as a companion test for treatment with pembrolizumab. This is qualitative assay using Monoclonal Mouse Anti-PD-L1, Clone 22C3 PD-L1 and EnVision FLEX visualization system on Autostainer Lin 48 to detect PD-L1 in FFPE samples, such as human non-small cell lung cancer tissue. Expression levels can be measured using the tumor proportion score (TPS), which measures the percentage of viable tumor cells showing partial or complete membrane staining at any intensity. Staining can show PD-L1 expression from 0% to 100%.

PD-L1 expression can also be detected using PD-L1 IHC 28-8 pharmDx, the FDA-approved in vitro diagnostic immunohistochemistry (IHC) test developed by Dako and Merck as a companion test for treatment with nivolumab. This qualitative assay uses the Monoclonal rabbit anti-PD-L1, Clone 28-8 and EnVision FLEX visualization system on Autostainer Lin 48 to detect PD-L1 in formalin-fixed, paraffin-embedded (FFPE) human cancer tissue.

Other commercially available tests for PD-L1 detection include the Ventana SP263 assay (developed by Ventana in collaboration with AstraZeneca) that utilizes monoclonal rabbit anti-PD-LI, Clone SP263 and the Ventana SP142 Assay (developed by Ventana in collaboration with Genentech/Roche) that uses rabbit monoclonal anti-PD-L1 clone SP142.

In some embodiments, a test approved by a regulatory authority, such as the US Food and Drug Administration (FDA), is used to determine the PD-L1 TPS of a cancer as disclosed herein. In various embodiment, the PD-L1 TPS is determined using a immunohistochemistry (IHC) test. In some embodiments, the IHC test is the PD-L1 IHC 22C3 pharmDx test. In various embodiments, the IHC test conducted with samples acquired by, for example, resection, CNB, or FNA.

In various embodiment, the patient has a PD-L1 TPS of less than 100%, 95%, 90%, 85%, 80%, 75%, 70%, 65%, 60%, 50%, 55%, 50%, 45%, 40%, 35%, 30%, 25%, 20%, 15%, 10%, 9%, 8%, 7%, 6%, 5%, 4%, 3%, 2%, or 1%. In various embodiments, the patient has a PD-L1 TPS of less than 50%, or less than 1%. In various embodiments, the patient has a PD-L1 TPS of more than or equal to 95%, 90%, 85%, 80%, 75%, 70%, 65%, 60%, 50%, 55%, 50%, 45%, 40%, 35%, 30%, 25%, 20%, 15%, 10%, 9%, 8%, 7%, 6%, 5%, 4%, 3%, 2%, or 1%. In various embodiments, the patient has a PD-L1 TPS of less than or equal to 100%, 95%, 90%, 85%, 80%, 75%, 70%, 65%, 60%, 50%, 55%, 50%, 45%, 40%, 35%, 30%, 25%, 20%, 15%, 10%, 9%, 8%, 7%, 6%, 5%, 4%, 3%, 2%, or 1%. In various embodiments, the patient has a PD-L1 TPS of less than or equal to 50%, or less than or equal to 1%. In various embodiments, the patient has a PD-L1 TPS of more than 95%, 90%, 85%, 80%, 75%, 70%, 65%, 60%, 50%, 55%, 50%, 45%, 40%, 35%, 30%, 25%, 20%, 15%, 10%, 9%, 8%, 7%, 6%, 5%, 4%, 3%, 2%, or 1%. In various embodiments, the patient has a PD-L1 TPS score a range bound by any of the values cited in the foregoing embodiments. For example, the patient has a PD-L1 TPS score in the range of less than 50% and more than or equal to 1%, less than or equal to 50% and more than 1%, less than or equal to 50% and more than or equal to 1%, or less than 50% and more than 1%.

In various embodiments, the patient has a PD-L1 TPS score in the range of less than 50% and more than or equal to 1%. In some embodiments, the patient has a PD-L1 TPS score in the range of more than or equal to 0% and less than 1%. In some embodiments, the patient has a PD-L1 TPS score in the range of more than 50% and less than or equal to 100%. In some embodiments, the patient has a PD-L1 TPS score of less than 1%. In some embodiments, the patient as a PD-L1 TPS score of 1-49%. In some embodiments, the patient has a PD-L1 TPS score of 50% or greater (i.e., 50%-100%).

### Efficacy of Treatment

The efficacy of the treatment methods described herein can be determined by the skilled clinician. However, a treatment is considered “effective treatment,” as the term is used herein, if one or more of the signs or symptoms of a condition described herein is altered in a beneficial manner, other clinically accepted symptoms are improved, or even ameliorated, or a desired response is induced e.g., by at least 10% following treatment according to the methods described herein. For example, in some embodiments, a 10% reduction in tumor volume observed in subjects receiving a formulation described herein would be considered to be an effective treatment. In some embodiments, tumor volume in the subject receiving treatment with a formulation described herein is reduced by least 10%, at least 11%, at least 12%, at least 13%, at least 14%, at least 15%, at least 16%, at least 17%, at least 18%, at least 19%, at least 20%, at least 30%, at least 31%, at least 32%, at least 33%, at least 34%, at least 35%, at least 36%, at least 37%, at least 38%, at least 40%, at least 45%, at least 50%, at least 55%, at least 60%, at least 65%, at least 70%, at least 75%, at least 80%, at least 85%, at least 90%, at least 95% or more compared to a subject that has not received the formulation.

The patient can respond to the sotorasib therapy as measured by at least a stable disease (SD), as determined by RECIST 1.1 protocol (Eisenhauer, et al., 2009). An at least stable disease is one that is a stable disease, has shown a partial response (PR) or has shown a complete response (CR) (i.e., “at least SD”=SD+PR+CR, often referred to as disease control). In various embodiments, the stable disease is neither sufficient shrinkage to qualify for partial response (PR) nor sufficient increase to qualify for progressive disease (PD). In various embodiments, the patient exhibits at least a partial response (i.e., “at least PR”=PR+CR, often referred to as objective response).

Response can be measured by one or more of decrease in tumor size, suppression or decrease of tumor growth, decrease in target or tumor lesions, delayed time to progression, no new tumor or lesion, a decrease in new tumor formation, an increase in survival or progression-free survival (PFS), and no metastases. In various embodiments, the progression of a patient's disease can be assessed by measuring tumor size, tumor lesions, or formation of new tumors or lesions, by assessing the patient using a computerized tomography (CT) scan, a positron emission tomography (PET) scan, a magnetic resonance imaging (MRI) scan, an X-ray, ultrasound, or some combination thereof.

Progression free survival can be assessed as described in the RECIST 1.1 protocol. In various embodiments, the patient exhibits a PFS of at least 3 months. In some embodiments, the patient exhibits a PFS of at least 6 months.

All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

### EMBODIMENTS

1. A formulation comprising
(a) sotorasib;
(b) a diluent in an amount of 40-95% (w/w),
(c) a disintegrant in an amount of 0.5-5% (w/w), and
(d) a lubricant in an amount of 0.25-5% (w/w).

2. The formulation of embodiment 1, comprising sotorasib in an amount of 1-50% (w/w).

3. The formulation of embodiment 1 or embodiment 2, wherein the diluent comprises one or more of lactose, dibasic calcium phosphate (DCP), mannitol, sorbitol, xylitol, calcium carbonate, magnesium carbonate, tribasic calcium phosphate, trehalose, microcrystalline cellulose, and starch.

4. The formulation of any one of embodiments 1-3, wherein the diluent comprises one or more of lactose, dibasic calcium phosphate (DCP), mannitol, microcrystalline cellulose, and starch.

5. The formulation of any one of embodiments 1-4, wherein the diluent comprises one or more of lactose and microcrystalline cellulose.

6. The formulation of any one of embodiments 1-4, wherein the diluent comprises one or more of lactose and starch.

7. The formulation of any one of embodiments 1-4, wherein the diluent comprises one or more of lactose, dibasic calcium phosphate (DCP), and mannitol.

8. The formulation of any one of embodiments 1-4 and 6, wherein starch is pregelatinized starch or corn starch.

9. The formulation of any one of embodiments 3-7, wherein lactose is lactose monohydrate.

10. The formulation of embodiment 1, comprising sotorasib in an amount of 1-20% (w/w).

11. The formulation of embodiment 10, comprising sotorasib in an amount of 1% (w/w).

12. The formulation of embodiment 10, comprising sotorasib in an amount of 20% (w/w).

13. The formulation of embodiment 10 or embodiment 12, comprising the diluent in an amount of 61-91% (w/w).

14. The formulation of embodiment 10 or embodiment 12, comprising the diluent in an amount of 68-84% (w/w).

15. The formulation of embodiment 10 or embodiment 12, comprising the diluent in an amount of 76% (w/w).

16. The formulation of any one of embodiments 10-15, wherein the diluent comprises a plastic diluent and a brittle diluent, wherein the ratio by weight of the plastic diluent to the brittle diluent ranges from 2.5:1 to 3.5:1.

17. The formulation of embodiments 10-15, wherein the diluent comprises a plastic diluent and a brittle diluent, wherein the ratio by weight of the plastic diluent to the brittle diluent ranges from 2.7:1 to 3.3:1.

18. The formulation of embodiments 10-15, wherein the diluent comprises a plastic diluent and a brittle diluent, wherein the ratio by weight of the plastic diluent to the brittle diluent is 3:1.

19. The formulation of embodiment 1, comprising sotorasib in an amount of 20-45% (w/w).

20. The formulation of embodiment 19, comprising sotorasib in an amount of 20% (w/w).

21. The formulation of embodiment 19, comprising sotorasib in an amount of 30% (w/w).

22. The formulation of embodiment 19, comprising sotorasib in an amount of 32% (w/w).

23. The formulation of embodiment 19, comprising sotorasib in an amount of 37.5% (w/w).

24. The formulation of embodiment 19, comprising sotorasib in an amount of 40% (w/w).

25. The formulation of embodiment 19 or embodiment 22, comprising the diluent in an amount of 51-77% (w/w)

26. The formulation of embodiment 19 or embodiment 22, comprising the diluent in an amount of 58-70% (w/w)

27. The formulation of embodiment 19 or embodiment 22, comprising the diluent in an amount of 64% (w/w).

28. The formulation of any one of embodiments 19-28, wherein the diluent comprises a plastic diluent and optionally a brittle diluent, wherein the ratio by weight of the plastic diluent to sotorasib and the brittle diluent, if present, taken together, ranges from 1.2:1 to 1.7:1.

29. The formulation of any one of embodiments 19-28, wherein the diluent comprises a plastic diluent and optionally a brittle diluent, wherein the ratio by weight of the plastic diluent to sotorasib and the brittle diluent, if present, taken together, ranges from 1.4:1 to 1.5:1.

30. The formulation of embodiment 1, comprising the diluent in an amount of 61-91% (w/w).

31. The formulation of embodiment 1, comprising the diluent in an amount of 68-84% (w/w).

32. The formulation of embodiment 1, comprising the diluent in an amount of 76% (w/w).

33. The formulation of embodiment 1, comprising the diluent in an amount of 51-77% (w/w).

34. The formulation of embodiment 1, comprising the diluent in an amount of 58-70% (w/w).

35. The formulation of embodiment 1, comprising the diluent in an amount of 64% (w/w).

36. The formulation of any one of embodiments 30-35, wherein diluent comprises a plastic diluent and optionally a brittle diluent, and wherein
(a) provided that the brittle diluent is present, the formulation is characterized by
   (1) a first ratio by weight of the plastic diluent to the brittle diluent that is greater than or equal to 2.5:1, 2.7:1, 3:1, 3.3:1, or 3.5:1; and
   (2) a second ratio by weight of the plastic diluent to sotorasib and the brittle diluent, taken together, is greater than or equal to 1.2:1, 1.4:1, 1.5:1, or 1.7:1 and less than the first ratio; or
(b) provided that the brittle diluent is absent, the formulation is characterized by a ratio by weight of the plastic diluent to sotorasib that is greater than or equal to 1.2:1, 1.4:1, 1.5:1, or 1.7:1 and less than 2.5:1, 2.7:1, 3:1, 3.3:1, or 3.5:1.

37. The formulation of embodiment 36, wherein the diluent comprises a plastic diluent and a brittle diluent, and wherein the first ratio is greater than or equal to 3:1 and the second ratio is greater than or equal to 1.4:1 and less than 3:1.

38. The formulation of any one of embodiments 30-35, wherein the diluent comprises a plastic diluent and no brittle diluent, and wherein the ratio by weight of the plastic diluent to sotorasib that is greater than or equal to 1.4:1 and less than 3:1.

39. The formulation of any one of embodiments 16-18, 28, 29, and 36-38, wherein the plastic diluent comprises one or more of microcrystalline cellulose and starch.
40. The formulation of embodiment 39, wherein the plastic diluent is microcrystalline cellulose.
41. The formulation of embodiment 39, wherein the plastic diluent is starch.
42. The formulation of embodiment 39 or embodiment 41, wherein starch is pregelatinized starch or corn starch.
43. The formulation of any one of embodiments 16-18, 28, 29, 36, and 37, wherein the brittle diluent comprises one or more of lactose, dibasic calcium phosphate (DCP), mannitol, sorbitol, xylitol, calcium carbonate, magnesium carbonate, tribasic calcium phosphate, and trehalose.
44. The formulation of embodiment 43, wherein the brittle diluent comprises one or more of lactose, dibasic calcium phosphate (DCP), or mannitol.
45. The formulation of embodiment 43, wherein the brittle diluent is lactose.
46. The formulation of any one of embodiments 43-45, wherein the lactose is lactose monohydrate.
47. The formulation of any one of embodiments 1-46, comprising a disintegrant in an amount of 1-5% (w/w).
48. The formulation of any one of embodiments 1-46, comprising a disintegrant in an amount of 3-5% (w/w).
49. The formulation of any one of embodiments 1-46, comprising a disintegrant in an amount of 2-4% (w/w).
50. The formulation of any one of embodiments 1-46, comprising a disintegrant in an amount of 3% (w/w).
51. The formulation of any one of embodiments 1 and 47-50, wherein the disintegrant comprises one or more of cross-linked sodium carboxy methyl cellulose (croscarmellose sodium), cross-linked polyvinylpyrrolidone (crospovidone), sodium starch glycolate, pregelatinized starch, calcium carboxymethyl cellulose, low substituted hydroxypropyl cellulose, and magnesium aluminum silicate.
52. The formulation of embodiment 51, wherein the disintegrant comprises one or more of croscarmellose sodium and sodium starch glycolate.
53. The formulation of embodiment 51, wherein the disintegrant is croscarmellose sodium.
54. The formulation of any one of embodiments 1-53, comprising a lubricant in an amount of 0.5-3% (w/w).
55. The formulation of any one of embodiments 1-53, comprising a lubricant in an amount of 0.5-1.5% (w/w).
56. The formulation of any one of embodiments 1-53, comprising a lubricant in an amount of 1% (w/w).
57. The formulation of any one of embodiments 1 and 54-56, wherein the lubricant comprises one or more of magnesium stearate, calcium stearate, oleic acid, caprylic acid, stearic acid, magnesium isovalerate, calcium laurate, magnesium palmitate, behenic acid, glyceryl behenate, glyceryl stearate, sodium stearyl fumarate, potassium stearyl fumarate, zinc stearate, sodium oleate, sodium stearate, sodium benzoate, sodium acetate, sodium chloride, talc, polyethylene glycol, and hydrogenated vegetable oil.
58. The formulation of embodiment 57, wherein the lubricant is magnesium stearate.
59. The formulation of any one of embodiments 1-58, comprising sotorasib in an amount of 1 mg to 360 mg.
60. The formulation of any one of embodiments 1-58, comprising sotorasib in an amount of 30 mg to 320 mg.
61. The formulation of any one of embodiments 1-58, comprising sotorasib in an amount of 1 mg.
62. The formulation of any one of embodiments 1-58, comprising sotorasib in an amount of 30 mg.
63. The formulation of any one of embodiments 1-58, comprising sotorasib in an amount of 120 mg.
64. The formulation of any one of embodiments 1-58, comprising sotorasib in an amount of 180 mg.
65. The formulation of any one of embodiments 1-58, comprising sotorasib in an amount of 240 mg.
66. The formulation of any one of embodiments 1-58, comprising sotorasib in an amount of 320 mg.
67. The formulation of any one of embodiments 1-58, comprising sotorasib in an amount of 360 mg.

68. The formulation of any one of embodiments 1-9, 51-53, 57, and 58, comprising sotorasib in an amount of 16-24% (w/w), a diluent in an amount of 61-91% (w/w), a disintegrant in an amount of 2.4-3.6% (w/w), and a lubricant in an amount of 0.8-1.2% (w/w).

69. The formulation of any one of embodiments 1-9, 51-53, 57, and 58, comprising sotorasib in an amount of 18-22% (w/w) sotorasib, a diluent in an amount of 68-84% (w/w), a disintegrant in an amount of 2.7-3.3% (w/w), and a lubricant in an amount of 0.9-1.1% (w/w).

70. The formulation of any one of embodiments 1-9, 51-53, 57, and 58, comprising sotorasib in an amount of 20% (w/w), a diluent in an amount of 76% (w/w), a disintegrant in an amount of 3% (w/w), and a lubricant in an amount of 1% (w/w).

71. The formulation of any one of embodiments 68-70, comprising sotorasib in an amount of 30 mg.

72. The formulation of any one of embodiments 68-70, comprising sotorasib in an amount of 120 mg.

73. The formulation of any one of embodiments 1-9, 51-53, 57, and 58, comprising sotorasib in an amount of 26-38% (w/w), a diluent in an amount of 51-77% (w/w), a disintegrant in an amount of 2.4-3.6% (w/w), and a lubricant in an amount of 0.8-1.2% (w/w).

74. The formulation of any one of embodiments 1-9, 51-53, 57, and 58, comprising sotorasib in an amount of 29-35% (w/w) sotorasib, a diluent in an amount of 58-70% (w/w), a disintegrant in an amount of 2.7-3.3% (w/w), and a lubricant in an amount of 0.9-1.1% (w/w).

75. The formulation of any one of embodiments 1-9, 51-53, 57, and 58, comprising sotorasib in an amount of 32% (w/w), a diluent in an amount of 64% (w/w), a disintegrant in an amount of 3% (w/w), and a lubricant in an amount of 1% (w/w).

76. The formulation of any one of embodiments 73-75, comprising sotorasib in an amount of 240 mg.

77. The formulation of any one of embodiments 73-75, comprising sotorasib in an amount of 320 mg.

78. The formulation of any one of embodiments 1-77, wherein the formulation is a solid dosage form.

79. The formulation of embodiment 78, wherein the solid dosage form is for oral administration.

80. The formulation of embodiment 78 or embodiment 79, wherein the solid dosage form is a tablet.

81. The formulation of embodiment 80, wherein the tablet is coated with a coating composition.

82. The formulation of embodiment 64, wherein the coating composition comprises polyvinyl alcohol.

83. The formulation of embodiment 82, wherein the coating composition further comprises one or more of titanium dioxide, polyethylene glycol, talc, and a coloring agent.

84. The formulation of any one of embodiment 1-83, wherein at least 50% of the sotorasib in the formulation is released within 30 minutes as measured by a dissolution test using a USP <711> apparatus 2 with 75 rpm paddle speed, at 37° C. in a dissolution medium of 900 ml of water at pH 6.7 comprising 50 mM sodium phosphate and a surfactant to maintain sink conditions.

85. The formulation of embodiment 84, wherein at least 80% of the sotorasib in the formulation is released within 30 minutes.

86. The formulation of embodiment 84, wherein at least 85% of the sotorasib in the formulation is released within 15 minutes.

87. The formulation of any one of embodiments 84-86, wherein the surfactant is 0.2-0.6% (w/v) sodium dodecyl sulfate (SDS).

88. The formulation of any one of embodiments 84-87, wherein the formulation comprises sotorasib in an amount of 120 mg and the dissolution medium comprises 0.5% (w/v) sodium dodecyl sulfate (SDS).

89. The formulation of any one of embodiments 84-87, wherein the formulation comprises sotorasib in an amount of 240 mg and the dissolution medium comprises 0.3% (w/v) sodium dodecyl sulfate (SDS).

90. The formulation of any one of embodiments 84-87, wherein the formulation comprises sotorasib in an amount of 320 mg and the dissolution medium comprises 0.4% (w/v) sodium dodecyl sulfate (SDS).

91. A formulation of any one of embodiments 1-90 for use as a medicament.

92. A formulation of any one of embodiments 1-90 for use in treating cancer.

93. A formulation of any one of embodiments 1-90 for use in treating cancer, wherein one or more cells of the cancer express a KRAS G12C mutant protein.

94. The formulation for use of embodiment 92 or embodiment 93, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

95. Use of the formulation of any one of embodiments 1-90 in the preparation of a medicament for treating cancer.

96. Use of the formulation of any one of embodiments 1-90 in the preparation of a medicament for treating cancer, wherein one or more cells of the cancer express a KRAS G12C mutant protein.

97. The use of embodiment 95 or 96, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

98. A method of treating cancer in a patient, the method comprising administering to the patient a therapeutically effective amount of sotorasib provided as the formulation of any one of embodiments 1-86, wherein the formulation provides the therapeutically effective amount in one or more dosage units.

99. The method of embodiment 98, wherein one or more cells of the cancer express a KRAS G12C mutant protein.

100. The method of embodiment 98 or embodiment 99, wherein the therapeutically effective amount is 180 mg, 240 mg, 320 mg, 360 mg, 720 mg, 960 mg.

101. The method of embodiment 98 or embodiment 99, wherein the therapeutically effective amount is 240 mg.

102. The method of embodiment 101, wherein the therapeutically effective amount is provided by the formulation of embodiment 63 or embodiment 72 in two dosage units.

103. The method of embodiment 101, wherein the therapeutically effective amount is provided by the formulation of embodiments 65 or embodiment 76 in one dosage unit.

104. The method of embodiment 98 or embodiment 99, wherein the therapeutically effective amount is 960 mg.

105. The method of embodiment 104, wherein the therapeutically effective amount is provided by the formulation of embodiment 63 or embodiment 72 in eight dosage units.

106. The method of embodiment 104, wherein the therapeutically effective amount is provided by the formulation of embodiments 65 or embodiment 76 in four dosage units.

107. The method of embodiment 104, wherein the therapeutically effective amount is provided by the formulation of embodiment 66 or embodiment 77 in three dosage units.

108. The method of any one of embodiments 98-107, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

109. The method of any one of embodiments 98-107, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

110. The method of embodiment 109, wherein the cancer is non-small cell lung cancer.

111. The method of embodiment 109, wherein the cancer is colorectal cancer.

112. The method of embodiment 109, wherein the cancer is pancreatic cancer.

113. The method of any one of embodiments 98-112, wherein the method further comprises dispersing the therapeutically effective amount provided as one or more dosage units in water by stirring before administration to the patient.

114. The method of embodiment 113, wherein the water is non-carbonated.

115. The method of embodiment 113 or embodiment 114, wherein the water has room-temperature.

116. The method of any one of embodiments 113-115, wherein the water has a volume of 120 mL.

117. The method of any one of embodiments 113-116, wherein the therapeutically effective amount is dispersed in water immediately or within 2 hours before administration to the patient.

118. The method of any one of embodiments 113-117, wherein the patient has difficulty swallowing solids.

### EXAMPLES

### Example 1

Dry granulation via roller compaction was selected as the manufacturing process to ensure adequate process and formulation performance, including flow, dose uniformity, and compressibility. Briefly, excipients including microcrystalline cellulose (MCC), lactose and croscarmellose sodium along with sotorasib were weighed and suspended in a blender for pre-blending. The pre-blend was passed through a suitable metal screen and subsequently mixed in a suitable tumble blender. Next, an appropriate quantity of screened magnesium stearate was dispensed to the pre-blend and mixed thoroughly in the blender at controlled duration and speed. The lubricated blend was then either directly compressed on a tablet press, slugged or, compacted into ribbons using a roll force and roll gap as shown in the table below. The ribbons and slugs were milled into granules an oscillating mill equipped with an 1.0 mm screen. Next, the obtained granules were lubricated by addition of screened magnesium stearate to the blender and thorough mixing at controlled duration and speed. The final blend was compressed into tablets on a tablet press. The tablet appearance, weight, thickness, and hardness were monitored at pre-defined intervals throughout the compression unit operation. Final tablets were coated, where noted in the following tables using suitable coating equipment.

| Formulation # | Roll Gap | Roll Force | |
|---|---|---|---|
| 1 | Slug¹ | Slug¹ | |
| 2 | 3 mm | 10 | kN/cm |
| 3 | 3 mm | 8 | kN/cm |
| 4 | 3 mm | 10 | kN/cm |
| 5 | 3 mm | 10 | kN/cm |
| 6 | 2 mm | 7 | kN/cm |
| 7 | 2 mm | 7 | kN/cm |
| 8 | 3 mm | 9 | kN/cm |
| 9a | 3 mm | 10 | kN/cm |
| 9b | 3 mm | 10 | kN/cm |
| 10a | 3 mm | 10 | kN/cm |
| 10b | 3 mm | 10 | kN/cm |

| | | |
|---|---|---|
| 11 | DC² | DC² |
| 12 | DC² | DC² |
| 13 | DC² | DC² |

¹Tablets were prepared by slugging the blends and then milling to a powder for tablet compression.
²Tablets were prepared by direct compression of the blend.

Formulations 1-13 (provided below in Tables 1-13) were prepared according to the methodology provided above.

TABLE 1
Formulation #1 (1% (w/w), 1 mg sotorasib)

| | | | |
|---|---|---|---|
| | | Theoretical | |
| | Percent | Quantity | |
| Component | (% w/w) | (mg/unit) | Function |

Intragranular

| | | | |
|---|---|---|---|
| Sotorasib | 1.00 | 1.00 | Active |
| Microcrystalline | 71.25 | 71.25 | Diluent |
| cellulose, PH102 | | | |
| Lactose monohydrate, | 23.75 | 23.75 | Diluent |
| Impalpable 313 | | | |
| Croscarmellose sodium | 3.00 | 3.00 | Disintegrant |
| Magnesium stearate, | 0.50 | 0.50 | Lubricant |
| Non-bovine | | | |

Extra Granular

| | | | |
|---|---|---|---|
| Magnesium stearate, | 0.50 | 0.50 | Lubricant |
| Non-bovine | | | |
| Core Tablet Total | 100.00 | 100.00 | |

TABLE 2
Formulation #2 (37.5% (w/w), 240 mg sotorasib)

| | | | |
|---|---|---|---|
| | | Theoretical | |
| | Percent | Quantity | |
| Component | (% w/w) | (mg/unit) | Function |

Intragranular

| | | | |
|---|---|---|---|
| Sotorasib | 37.50 | 240.00 | Active |
| Microcrystalline | 44.00 | 281.60 | Diluent |
| cellulose, PH102 | | | |
| Lactose monohydrate, | 14.50 | 92.80 | Diluent |
| Impalpable 313 | | | |
| Croscarmellose sodium | 3.00 | 19.20 | Disintegrant |
| Magnesium stearate, | 0.50 | 3.20 | Lubricant |
| Non-bovine | | | |

Extra Granular

| | | | |
|---|---|---|---|
| Magnesium stearate, | 0.50 | 3.20 | Lubricant |
| Non-bovine | | | |
| Core Tablet Total | 100.00 | 640.00 | |

TABLE 3
Formulation #3 (50% (w/w), 360 mg sotorasib)

| | | | |
|---|---|---|---|
| | | Theoretical | |
| | Percent | Quantity | |
| Component | (% w/w) | (mg/unit) | Function |

Intragranular

| | | | |
|---|---|---|---|
| Sotorasib | 50.00 | 360.00 | Active |
| Microcrystalline | 34.50 | 248.40 | Diluent |
| cellulose, PH102 | | | |
| Lactose monohydrate, | 11.50 | 82.80 | Diluent |
| Impalpable 313 | | | |
| Croscarmellose sodium | 3.00 | 21.60 | Disintegrant |
| Magnesium stearate, | 0.50 | 3.60 | Lubricant |
| Non-bovine | | | |

Extra Granular

| | | | |
|---|---|---|---|
| Magnesium stearate, | 0.50 | 3.60 | Lubricant |
| Non-bovine | | | |
| Core Tablet Total | 100.00 | 720.00 | |

TABLE 4
Formulation #4 (30% (w/w), 180 mg sotorasib)

| | | | |
|---|---|---|---|
| | | Theoretical | |
| | Percent | Quantity | |
| Component | (% w/w) | (mg/unit) | Function |

Intragranular

| | | | |
|---|---|---|---|
| Sotorasib | 30.00 | 180.00 | Active |
| Microcrystalline | 57.00 | 342.00 | Diluent |
| cellulose, PH102 | | | |
| Lactose monohydrate, | 9.00 | 54.00 | Diluent |
| Impalpable 313 | | | |
| Croscarmellose sodium | 3.00 | 18.00 | Disintegrant |
| Magnesium stearate, | 0.50 | 3.00 | Lubricant |
| Non-bovine | | | |

Extra Granular

| | | | |
|---|---|---|---|
| Magnesium stearate, | 0.50 | 3.00 | Lubricant |
| Non-bovine | | | |
| Core Tablet Total | 100.00 | 600.00 | |

Extra Granular

TABLE 5
Formulation #5 (40% (w/w), 360 mg sotorasib)

| | | | |
|---|---|---|---|
| | | Theoretical | |
| | Percent | Quantity | |
| Component | (% w/w) | (mg/unit) | Function |

Intragranular

| | | | |
|---|---|---|---|
| Sotorasib | 40.00 | 360.00 | Active |
| Microcrystalline | 56.00 | 504.00 | Diluent |
| cellulose, PH102 | | | |
| Lactose monohydrate, | 0.00 | 0.00 | Diluent |
| Impalpable 313 | | | |
| Croscarmellose sodium | 3.00 | 27.00 | Disintegrant |
| Magnesium stearate, | 0.50 | 4.50 | Lubricant |
| Non-bovine | | | |

Extra Granular

| | | | |
|---|---|---|---|
| Magnesium stearate, | 0.50 | 4.50 | Lubricant |
| Non-bovine | | | |
| Core Tablet Total | 100.00 | 900.00 | |

TABLE 6
Formulation #6 (20% (w/w), 30 mg sotorasib)

| | | | |
|---|---|---|---|
| | | Theoretical | |
| | Percent | Quantity | |
| Component | (% w/w) | (mg/unit) | Function |

Intragranular

| | | | |
|---|---|---|---|
| Sotorasib | 20.00 | 30.00 | Active |
| Microcrystalline | 57.00 | 85.50 | Diluent |
| cellulose, PH102 | | | |
| Lactose monohydrate, | 19.00 | 28.50 | Diluent |
| Impalpable 313 | | | |
| Croscarmellose sodium | 3.00 | 4.50 | Disintegrant |
| Magnesium stearate, | 0.50 | 0.75 | Lubricant |
| Non-bovine | | | |

Extra Granular

| | | | |
|---|---|---|---|
| Magnesium stearate, | 0.50 | 0.75 | Lubricant |
| Non-bovine | | | |
| Core Tablet Total | 100.00 | 150.00 | |

TABLE 7
Formulation #7 (20% (w/w), 120 mg sotorasib)

| | | | |
|---|---|---|---|
| | | Theoretical | |
| | Percent | Quantity | |
| Component | (% w/w) | (mg/unit) | Function |

Intragranular

| | | | |
|---|---|---|---|
| Sotorasib | 20.00 | 120.00 | Active |
| Microcrystalline | 57.00 | 342.00 | Diluent |
| cellulose, PH102 | | | |
| Lactose monohydrate, | 19.00 | 114.00 | Diluent |
| Impalpable 313 | | | |
| Croscarmellose sodium | 3.00 | 18.00 | Disintegrant |
| Magnesium stearate, | 0.50 | 3.00 | Lubricant |
| Non-bovine | | | |

Extra Granular

| | | | |
|---|---|---|---|
| Magnesium stearate, | 0.50 | 3.00 | Lubricant |
| Non-bovine | | | |
| Core Tablet Total | 100.00 | 600.00 | |

TABLE 8
Formulation #8 (20% (w/w), 120 mg sotorasib)

| | | | |
|---|---|---|---|
| | | Theoretical | |
| | Percent | Quantity | |
| Component | (% w/w) | (mg/unit) | Function |

Intragranular

| | | | |
|---|---|---|---|
| Sotorasib | 20.00 | 120.00 | Active |
| Microcrystalline | 57.00 | 342.00 | Diluent |
| cellulose, PH102 | | | |
| Lactose monohydrate, | 19.00 | 114.00 | Diluent |
| Impalpable 313 | | | |
| Croscarmellose | 3.00 | 18.00 | Disintegrant |
| sodium | | | |
| Magnesium stearate, | 0.50 | 3.00 | Lubricant |
| Non-bovine | | | |

Extra Granular

| | | | |
|---|---|---|---|
| Magnesium stearate, | 0.50 | 3.00 | Lubricant |
| Non-bovine | | | |
| Core Tablet Total | 100.00 | 600.00 | |

Film Coating

| | | | |
|---|---|---|---|
| Opadry ® II | 3.00 | 18.00 | Coating Material |
| Yellow 85F120132 | | | |
| Purified water | — | — | Coating Solvent |
| Total | 103.00 | 618.00 | |

TABLE 9a
Formulation #9a (32% (w/w), 240 mg sotorasib)

| | | | |
|---|---|---|---|
| | | Theoretical | |
| | Percent | Quantity | |
| Component | (% w/w) | (mg/unit) | Function |

Intragranular

| | | | |
|---|---|---|---|
| Sotorasib | 32.00 | 240.0 | Active |
| Microcrystalline | 57.00 | 427.5 | Diluent |
| cellulose, PH102 | | | |
| Lactose monohydrate, | 7.00 | 52.50 | Diluent |
| Impalpable 313 | | | |
| Croscarmellose | 3.00 | 22.50 | Disintegrant |
| sodium | | | |
| Magnesium stearate, | 0.50 | 3.750 | Lubricant |
| Non-bovine | | | |

Extra Granular

| | | | |
|---|---|---|---|
| Magnesium stearate, | 0.50 | 3.750 | Lubricant |
| Non-bovine | | | |
| Core Tablet Total | 100.0 | 750.0 | |

Film Coating

| | | | |
|---|---|---|---|
| Opadry ® II | 3.00 | 22.50 | Coating Material |
| Yellow 85F120132 | | | |
| Purified water | — | — | Coating Solvent |
| Total | 103.00 | 772.5 | |

TABLE 9b
Formulation #9b (32% (w/w), 240 mg sotorasib)

| | | | |
|---|---|---|---|
| | | Theoretical | |
| | Percent | Quantity | |
| Component | (% w/w) | (mg/unit) | Function |

Intragranular

| | | | |
|---|---|---|---|
| Sotorasib | 32.00 | 240.0 | Active |
| Microcrystalline | 57.00 | 427.5 | Diluent |
| cellulose, PH102 | | | |
| Lactose monohydrate, | 7.00 | 52.50 | Diluent |
| Impalpable 313 | | | |
| Croscarmellose | 3.00 | 22.50 | Disintegrant |
| sodium | | | |
| Magnesium stearate, | 0.50 | 3.750 | Lubricant |
| Non-bovine | | | |

Extra Granular

| | | | |
|---|---|---|---|
| Magnesium stearate, | 0.50 | 3.750 | Lubricant |
| Non-bovine | | | |
| Core Tablet Total | 100.0 | 750.0 | |

Film Coating

| | | | |
|---|---|---|---|
| Opadry ® II | 3.00 | 22.50 | Coating Material |
| Yellow 85F120222-CN | | | |
| Purified water | — | — | Coating Solvent |
| Total | 103.00 | 772.5 | |

TABLE 10a
Formulation #10a (32% (w/w), 320 mg sotorasib, Batch (a))

| | | | |
|---|---|---|---|
| | | Theoretical | |
| | Percent | Quantity | |
| Component | (% w/w) | (mg/unit) | Function |

Intragranular

| | | | |
|---|---|---|---|
| Sotorasib | 32.00 | 320.0 | Active |
| Microcrystalline | 57.00 | 570.0 | Diluent |
| cellulose, PH102 | | | |
| Lactose monohydrate, | 7.00 | 70.00 | Diluent |
| Impalpable 313 | | | |
| Croscarmellose | 3.00 | 30.00 | Disintegrant |
| sodium | | | |
| Magnesium stearate, | 0.500 | 5.000 | Lubricant |
| Non-bovine | | | |

Extra Granular

| | | | |
|---|---|---|---|
| Magnesium stearate, | 0.500 | 5.00 | Lubricant |
| Non-bovine | | | |
| Core Tablet Total | 100.00 | 1000 | |

Film Coating

| | | | |
|---|---|---|---|
| Opadry ® II | 3.00 | 30.00 | Coating Material |
| Beige 85F170037 | | | |
| Purified water | — | — | Coating Solvent |
| Total | 103.00 | 1030 | |

TABLE 10b
Formulation #10b (32% (w/w), 320 mg sotorasib, Batch (b))

| | | | |
|---|---|---|---|
| | | Theoretical | |
| | Percent | Quantity | |
| Component | (% w/w) | (mg/unit) | Function |

Intragranular

| | | | |
|---|---|---|---|
| Sotorasib | 32.00 | 320.0 | Active |
| Microcrystalline cellulose, | 57.00 | 570.0 | Diluent |
| PH102 | | | |
| Lactose monohydrate, | 7.00 | 70.00 | Diluent |
| Impalpable 313 | | | |
| Croscarmellose sodium | 3.00 | 30.00 | Disintegrant |
| Magnesium stearate, Non-bovine | 0.500 | 5.000 | Lubricant |

Extra Granular

| | | | |
|---|---|---|---|
| Magnesium stearate, Non-bovine | 0.500 | 5.00 | Lubricant |
| Core Tablet Total | 100.00 | 1000 | |
| Film Coating | | | |
| Opadry ® II | 3.00 | 30.00 | Coating |
| Beige 85F170037 | | | Material |
| Purified water | — | — | Coating |
| | | | Solvent |
| Total | 103.00 | 1030 | |

TABLE 11
Formulation #11 (20% (w/w), 120 mg sotorasib)

| | | | |
|---|---|---|---|
| | | Theoretical | |
| | Percent | Quantity | |
| Component | (% w/w) | (mg/unit) | Function |

Intragranular

| | | | |
|---|---|---|---|
| Sotorasib | 20.00 | 120.00 | Active |
| Microcrystalline cellulose, | 57.00 | 342.00 | Diluent |
| PH102 | | | |
| Dibasic calcium phosphate | 19.00 | 114.00 | Diluent |
| (DCP) | | | |
| Croscarmellose sodium | 3.00 | 18.00 | Disintegrant |
| Magnesium stearate, Non-bovine | 0.50 | 3.00 | Lubricant |

Extra Granular

| | | | |
|---|---|---|---|
| Magnesium stearate, Non-bovine | 0.50 | 3.00 | Lubricant |
| Core Tablet Total | 100.00 | 600.00 | |

TABLE 12
Formulation #12 (20% (w/w), 120 mg sotorasib)

| | | | |
|---|---|---|---|
| | | Theoretical | |
| | Percent | Quantity | |
| Component | (% w/w) | (mg/unit) | Function |

Intragranular

| | | | |
|---|---|---|---|
| Sotorasib | 20.00 | 120.00 | Active |
| Microcrystalline cellulose, | 57.00 | 342.00 | Diluent |
| PH102 | | | |
| Mannitol | 19.00 | 114.00 | Diluent |
| Croscarmellose sodium | 3.00 | 18.00 | Disintegrant |
| Magnesium stearate, Non-bovine | 0.50 | 3.00 | Lubricant |

Extra Granular

| | | | |
|---|---|---|---|
| Magnesium stearate, Non-bovine | 0.50 | 3.00 | Lubricant |
| Core Tablet Total | 100.00 | 600.00 | |

TABLE 13
Formulation #13 (20% (w/w), 120 mg sotorasib)

| | | | |
|---|---|---|---|
| | | Theoretical | |
| | Percent | Quantity | |
| Component | (% w/w) | (mg/unit) | Function |

Intragranular

| | | | |
|---|---|---|---|
| Sotorasib | 20.00 | 120.00 | Active |
| Starch | 57.00 | 342.00 | Diluent |
| Lactose monohydrate | 19.00 | 114.00 | Diluent |
| Sodium starch glycolate (SSG) | 3.00 | 18.00 | Disintegrant |
| Magnesium stearate, Non-bovine | 0.50 | 3.00 | Lubricant |

Extra Granular

| | | | |
|---|---|---|---|
| Magnesium stearate, Non-bovine | 0.50 | 3.00 | Lubricant |
| Core Tablet Total | 100.00 | 600.00 | |

### Example 2—Stability Studies

Sotorasib 120 mg (Formulations #7 and #8) tablets, sotorasib 240 mg (Formulation #9b), sotorasib 320 mg (Formulation #10b), and sotorasib 30 mg (Formulation #6) tablets were packaged into 75 cc (with silica gel as the desiccant) or 215 cc HDPE (high density polyethylene) bottles (without desiccant), heat induction seal and polypropylene child resistant closure. The bottled tablets were placed on stability at −20° C., 5° C., the long-term storage condition of 30° C./65% RH (relative humidity), and the accelerated condition of 40° C./75% RH. Samples were evaluated for water content, assay (% label claim), total impurities and dissolution. The water content was determined by Karl Fischer volumetric titration in a titration vessel filled with methanol, where accurately weighed tablets were homogenized in situ with a homogenizer and titrated with standardized KF titrant. Assay (% label claim) was determined using a reversed phase HPLC method with UV detection. The primary analyte was separated from related impurities and potential degradants by gradient elution and quantified against an external reference standard of known purity. The sum of organic impurities, whose levels were determined using the same method as assay determination are reported as the total impurities. See Tables 14-28 for results from stability studies.

TABLE 14
Stability data (Formulation #8: 20% (w/w), 120 mg sotorasib) at 5°
C. Tablets were packaged into the 30 count 75 cc HDPE (high density polyethylene)
bottles with silica gel as desiccant, heat induction seal and polypropylene
child resistant closure and placed on stability at conditions specified below.

| | Acceptance | Time (Months) | | |
|---|---|---|---|---|
| | Criteria | Initial | 3 | 6 |
| Description | Yellow, oblong, | Yellow, oblong, | Yellow, oblong, | Yellow, oblong, |
| | debossed tablet | debossed tablet | debossed tablet | debossed tablet |
| | with “AMGN” on | with “AMGN” on | with “AMGN” on | with “AMGN” on |
| | one side and | one side and | one side and | one side and |
| | “120” on the | “120” on the | “120” on the | “120” on the |
| | opposite side. | opposite side. | opposite side. | opposite side. |
| | No obvious | No obvious | No obvious | No obvious |
| | physical defects | physical defects | physical defects | physical defects |
| Assay (% Label Claim) | 90.0 to 110.0 | 99.8 | 99.5 | 98.2 |
| Total impurities | ≤4.0 | 0.47 | 0.45 | 0.46 |
| Water Content (w/w %) | Report | 3.6 | 3.3 | 3.3 |
| Dissolution | | Mean, Min, | Mean, Min, | Mean, Min, |
| (% Dissolved)^{a} | | Max | Max | Max |
| 15 min | Report | 99, 94, 101 | 98, 95, 100 | 94, 93, 95 |
| 30 min | | 101, 99, 102 | 100, 97, 102 | 97, 95, 99 |
| 45 min | | 101, 101, 102 | 100, 98, 101 | 98, 96, 99 |
| 60 min | | 101, 101, 102 | 100, 98, 101 | 98, 96, 99 |

^{a}For experimental conditions see Example 3.

TABLE 15
Stability data (Formulation #8: 20% (w/w), 120 mg sotorasib) at 30° C./65%
RH. Tablets were packaged into the 30 count 75 cc HDPE (high density polyethylene)
bottles with silica gel as desiccant, heat induction seal and polypropylene child
resistant closure and placed on stability at conditions specified below.

| | Acceptance | Time (Months) | | |
|---|---|---|---|---|
| | Criteria | Initial | 3 | 6 |
| Description | Yellow, oblong, | Yellow, oblong, | Yellow, oblong, | Yellow, oblong, |
| | debossed tablet | debossed tablet | debossed tablet | debossed tablet |
| | with “AMGN” on | with “AMGN” on | with “AMGN” on | with “AMGN” on |
| | one side and | one side and | one side and | one side and |
| | “120” on the | “120” on the | “120” on the | “120” on the |
| | opposite side. | opposite side. | opposite side. | opposite side. |
| | No obvious | No obvious | No obvious | No obvious |
| | physical defects | physical defects | physical defects | physical defects |
| Assay (% Label | 90.0 to 110.0 | 99.8 | 98.9 | 99.6 |
| Claim) | | | | |
| Total impurities | ≤4.0 | 0.47 | 0.45 | 0.46 |
| Water Content | Report | 3.6 | 3.2 | 3.4 |
| (w/w %) | | | | |
| Dissolution | | Mean, Min, | Mean, Min, | Mean, Min, |
| (% Dissolved)^{a} | | Max | Max | Max |
| 15 min | Report | 99, 94, 101 | 97, 93, 101 | 95, 92, 97 |
| 30 min | | 101, 99, 102 | 99, 96, 101 | 97, 95, 100 |
| 45 min | | 101, 101, 102 | 99, 96, 101 | 97, 96, 100 |
| 60 min | | 101, 101, 102 | 99, 97, 102 | 97, 96, 100 |

^{a}For experimental conditions see Example 3.

TABLE 16
Stability data (Formulation #8: 20% (w/w), 120 mg sotorasib) at 40° C./75% RH. Tablets were packaged
into the 30 count 75 cc HDPE (high density polyethylene) bottles with silica gel as desiccant, heat induction
seal and polypropylene child resistant closure and placed on stability at conditions specified below.

| | Acceptance | Time (Months) | | | |
|---|---|---|---|---|---|
| Test | Criteria | Initial | 1 | 3 | 6 |
| Description | Yellow, oblong, | Yellow, oblong, | Yellow, oblong, | Yellow, oblong, | Yellow, oblong, |
| | debossed tablet | debossed tablet | debossed tablet | debossed tablet | debossed tablet |
| | with “AMGN” on | with “AMGN” on | with “AMGN” on | with “AMGN” on | with “AMGN” on |
| | one side and | one side and | one side and | one side and | one side and |
| | “120” on the | “120” on the | “120” on the | “120” on the | “120” on the |
| | opposite side. | opposite side. | opposite side. | opposite side. | opposite side. |
| | No obvious | No obvious | No obvious | No obvious | No obvious |
| | physical defects | physical defects | physical defects | physical defects | physical defects |
| Assay (% Label | 90.0 to 110.0 | 99.8 | 99.5 | 99.4 | 97.6 |
| Claim) | | | | | |
| Total impurities | ≤4.0 | 0.47 | 0.45 | 0.45 | 0.50 |
| Water Content | Report | 3.6 | 3.3 | 3.3 | 3.7 |
| (w/w %) | | | | | |
| Dissolution | | Mean, Min, | Mean, Min, | Mean, Min, | Mean, Min, |
| (% Dissolved)^{a} | | Max | Max | Max | Max |
| 15 min | Report | 99, 94, 101 | 96, 94, 97 | 96, 93, 100 | 93, 91, 95 |
| 30 min | | 101, 99, 102 | 99, 97, 100 | 98, 96, 101 | 97, 96, 97 |
| 45 min | | 101, 101, 102 | 100, 97, 101 | 99, 96, 101 | 97, 97, 98 |
| 60 min | | 101, 101, 102 | 100, 98, 101 | 99, 97, 101 | 97, 97, 98 |

^{a}For experimental conditions see Example 3.

TABLE 17
Stability data (Formulation #9b: 32% (w/w), 240 mg
sotorasib) at −20° C. Tablets were packaged into
the 20 count 75 cc HDPE (high density polyethylene) bottles,
heat induction seal and polypropylene child resistant
closure and placed on stability at conditions specified below.

| | Acceptance | Time (Months) | |
|---|---|---|---|
| Test | Criteria | Initial | 1 |
| Description | Yellow, oval | Yellow, oval | Yellow, oval |
| | debossed tablet | debossed tablet | debossed tablet |
| | with “AMG” | with “AMG” | with “AMG” |
| | on one side and | on one side and | on one side and |
| | “240” on the | “240” on the | “240” on the |
| | opposite side. | opposite side. | opposite side. |
| | No obvious | No obvious | No obvious |
| | physical | physical | physical |
| | defects | defects | defects |
| Assay (% | 90.0 to 110.0 | 99.4 | 99.7 |
| Label Claim) | | | |
| Total impurities | ≤4.0 | <0.05 | <0.05 |
| Water Content | Report | 3.3 | 3.2 |
| (w/w %) | | | |
| Dissolution | Report | Mean, Min, | Mean, Min, |
| (% Dissolved)^{a} | | Max | Max |
| 15 min | | 97, 95, 99 | 97, 95, 99 |
| 30 min | | 100, 99, 101 | 100, 98, 101 |
| 45 min | | 101, 100, 102 | 101, 99, 102 |
| 60 min | | 101, 100, 102 | 101, 100, 102 |

^{a}For experimental conditions see Example 3.

TABLE 18
Stability data (Formulation #9b: 32% (w/w), 240 mg sotorasib) at 30°
C./65% RH. Tablets were packaged into the 30 count 75 cc HDPE (high density
polyethylene) bottles, heat induction seal and polypropylene child resistant
closure and placed on stability at conditions specified below.

| | Acceptance | Time (Months) | | |
|---|---|---|---|---|
| Test | Criteria | Initial | 2 | 6 |
| Description | Yellow, oval | Yellow, oval | Yellow, oval | Yellow, oval |
| | debossed tablet | debossed tablet | debossed tablet | debossed tablet |
| | with “AMG” on | with “AMG” on | with “AMG” on | with “AMG” on |
| | one side and | one side and | one side and | one side and |
| | “240” on the | “240” on the | “240” on the | “240” on the |
| | opposite side. | opposite side. | opposite side. | opposite side. |
| | No obvious | No obvious | No obvious | No obvious |
| | physical defects | physical defects | physical defects | physical defects |
| Assay (% Label | 90.0 to 110.0 | 99.4 | 99.5 | 99.1 |
| Claim) | | | | |
| Total impurities | ≤4.0 | <0.05 | <0.05 | <0.05 |
| Water Content | Report | 3.3 | 3.4 | 3.3 |
| (w/w %) | | | | |
| Dissolution | | Mean, Min, | Mean, Min, | Mean, Min, |
| (% Dissolved)^{a} | | Max | Max | Max |
| 15 min | Report | 97, 95, 99 | 93, 92, 94 | 94, 91, 97 |
| 30 min | | 100, 99, 101 | 96, 95, 97 | 99, 97, 102 |
| 45 min | | 101, 100, 102 | 98, 96, 99 | 100, 98,102 |
| 60 min | | 101, 100, 102 | 98, 96, 99 | 100, 98, 102 |

^{a}For experimental conditions see Example 3.

TABLE 19
Stability data (Formulation #9b: 32% (w/w), 240 mg sotorasib) at 40° C./75% RH. Tablets
were packaged into the 30 count 75 cc HDPE (high density polyethylene) bottles, heat induction
seal and polypropylene child resistant closure and placed on stability at conditions specified below.

| | Acceptance | Time (Months) | | | |
|---|---|---|---|---|---|
| Test | Criteria | Initial | 1 | 3 | 6 |
| Description | Yellow, oval | Yellow, oval | Yellow, oval | Yellow, oval | Yellow, oval |
| | debossed tablet | debossed tablet | debossed tablet | debossed tablet | debossed tablet |
| | with “AMG” on | with “AMG” on | with “AMG” on | with “AMG” on | with “AMG” on |
| | one side and | one side and | one side and | one side and | one side and |
| | “240” on the | “240” on the | “240” on the | “240” on the | “240” on the |
| | opposite side. | opposite side. | opposite side. | opposite side. | opposite side. |
| | No obvious | No obvious | No obvious | No obvious | No obvious |
| | physical defects | physical defects | physical defects | physical defects | physical defects |
| Assay (% Label | 90.0 to 110.0 | 99.4 | 100.0 | 99.9 | 99.3 |
| Claim) | | | | | |
| Total impurities | ≤4.0 | >0.05 | >0.05 | 0.09 | 0.09 |
| Water Content | Report | 3.3 | 3.3 | 3.6 | 3.3 |
| (w/w %) | | | | | |
| Dissolution | | Mean, Min, | Mean, Min, | Mean, Min, | Mean, Min, |
| (% Dissolved)^{a} | | Max | Max | Max | Max |
| 15 min | Report | 97, 95, 99 | 94, 92, 96 | 95, 92, 96 | 95, 94, 96 |
| 30 min | | 100, 99, 101 | 99, 97, 101 | 99, 98, 100 | 100, 100, 101 |
| 45 min | | 101, 100, 102 | 100, 99, 101 | 100, 99, 101 | 101, 101, 102 |
| 60 min | | 101, 100, 102 | 100, 99, 102 | 101, 99, 102 | 101, 99, 102 |

^{a}For experimental conditions see Example 3.

TABLE 20
Stability data (Formulation #10b: 32% (w/w), 320 mg sotorasib) at
5° C. Tablets were packaged into the 90 count 215 cc HDPE (high
density polyethylene) bottles, heat induction seal and polypropylene
child resistant closure and placed on stability at conditions specified below.

| | Acceptance | Time (Months) | | |
|---|---|---|---|---|
| | Criteria | Initial | 3 | 6 |
| Description | Beige, oval | Beige, oval | Beige, oval | Beige, oval |
| | tablet debossed | tablet debossed | tablet debossed | tablet debossed |
| | with “AMG” on | with “AMG” on | with “AMG” on | with “AMG” on |
| | one side and | one side and | one side and | one side and |
| | “320” on the | “320” on the | “320” on the | “320” on the |
| | opposite side. | opposite side. | opposite side. | opposite side. |
| | No obvious | No obvious | No obvious | No obvious |
| | physical defects. | physical defects. | physical defects. | physical defects. |
| Assay (% Label | 90.0 to 110.0 | 99.3 | 99.7 | 100.3 |
| Claim) | | | | |
| Total impurities | ≤2.0 | <0.05 | 0.05 | 0.11 |
| Water Content | ≤8.0 | 2.9 | 3.0 | 2.9 |
| (w/w %) | | | | |
| Dissolution | | Mean, Min, | Mean, Min, | Mean, Min, |
| (% Dissolved)^{a} | | Max | Max | Max |
| 15 min | Report | 96, 95, 98 | 101, 98, 107 | 96, 94, 98 |
| 30 min | | 99, 98, 101 | 102, 101, 103 | 99, 97, 101 |
| 45 min | | 100, 99, 102 | 102, 101, 105 | 99, 97, 102 |
| 60 min | | 100, 99, 102 | 103, 101, 104 | 100, 97, 102 |

^{a}For experimental conditions see Example 3.

TABLE 21
Stability data (Formulation #10b: 32% (w/w), 320 mg sotorasib) at 30°
C./65% RH. Tablets were packaged into the 90 count 215 cc HDPE (high density
polyethylene) bottles, heat induction seal and polypropylene child resistant
closure and placed on stability at conditions specified below.

| | Acceptance | Time (Months) | | |
|---|---|---|---|---|
| | Criteria | Initial | 3 | 6 |
| Description | Beige, oval | Beige, oval | Beige, oval | Beige, oval |
| | tablet debossed | tablet debossed | tablet debossed | tablet debossed |
| | with “AMG” on | with “AMG” on | with “AMG” on | with “AMG” on |
| | one side and | one side and | one side and | one side and |
| | “320” on the | “320” on the | “320” on the | “320” on the |
| | opposite side. | opposite side. | opposite side. | opposite side. |
| | No obvious | No obvious | No obvious | No obvious |
| | physical defects. | physical defects. | physical defects. | physical defects. |
| Assay (% Label | 90.0 to 110.0 | 99.3 | 99.5 | 100.2 |
| Claim) | | | | |
| Total impurities | ≤2.0 | <0.05 | 0.06 | 0.06 |
| Water Content | ≤8.0 | 2.9 | 3.0 | 2.9 |
| (w/w %) | | | | |
| Dissolution | | Mean, Min, | Mean, Min, | Mean, Min, |
| (% Dissolved)^{a} | | Max | Max | Max |
| 15 min | Report | 96, 95, 98 | 99, 97, 102 | 95, 93, 96 |
| 30 min | | 99, 98, 101 | 102, 100, 104 | 98, 96, 98 |
| 45 min | | 100, 99, 102 | 102, 101, 104 | 98, 97, 99 |
| 60 min | | 100, 99, 102 | 102, 101, 104 | 99, 97, 100 |

^{a}For experimental conditions see Example 3.

TABLE 22
Stability data (Formulation #10b: 32% (w/w), 320 mg sotorasib) at 40° C./75% RH. Tablets
were packaged into the 90 count 215 cc HDPE (high density polyethylene) bottles, heat induction
seal and polypropylene child resistant closure and placed on stability at conditions specified below.

| | Acceptance | Time (Months) | | | |
|---|---|---|---|---|---|
| Test | Criteria | Initial | 1 | 3 | 6 |
| Description | Beige, oval | Beige, oval | Beige, oval | Beige, oval | Beige, oval |
| | tablet debossed | tablet debossed | tablet debossed | tablet debossed | tablet debossed |
| | with “AMG” on | with “AMG” on | with “AMG” on | with “AMG” on | with “AMG” on |
| | one side and | one side and | one side and | one side and | one side and |
| | “320” on the | “320” on the | “320” on the | “320” on the | “320” on the |
| | opposite side. | opposite side. | opposite side. | opposite side. | opposite side. |
| | No obvious | No obvious | No obvious | No obvious | No obvious |
| | physical defects. | physical defects. | physical defects. | physical defects. | physical defects. |
| Assay (% Label | 90.0 to 110.0 | 99.4 | 98.5 | 100.9 | 99.7 |
| Claim) | | | | | |
| Total impurities | ≤2.0 | <0.05 | <0.05 | 0.05 | <0.05 |
| Water Content | ≤8.0 | 2.9 | 2.9 | 3.0 | 3.0 |
| (w/w %) | | | | | |
| Dissolution | | Mean, Min, | Mean, Min, | Mean, Min, | Mean, Min, |
| (% Dissolved)^{a} | | Max | Max | Max | Max |
| 15 min | Report | 96, 95, 98 | 98, 96, 100 | 98, 96, 100 | 94, 92, 96 |
| 30 min | | 99, 98, 101 | 100, 98, 104 | 101, 101, 103 | 97, 96, 99 |
| 45 min | | 100, 99, 102 | 103, 101, 105 | 102, 101, 103 | 98, 96, 99 |
| 60 min | | 100, 99, 102 | 100, 99, 101 | 102, 101, 103 | 98, 97, 100 |

^{a}For experimental conditions see Example 3.

TABLE 23
Stability data (Formulation #7: 20% (w/w), 120 mg sotorasib, uncoated) at
5° C. Tablets were packaged into the 30 count 75 cc HDPE (high density polyethylene)
bottles with silica gel as desiccant, heat induction seal and polypropylene child
resistant closure and placed on stability at conditions specified below.

| | Acceptance | Time (Months) | | | |
|---|---|---|---|---|---|
| Test | Criteria | Initial | 1 | 3 | 6 |
| Description | White to off-white | Conforms | Conforms | Conforms | Conforms^{a} |
| | round tablets with no | | | | |
| | obvious physical defects | | | | |
| Assay (% Label | 90.0 to 110.0 | 102.1 | 102.7 | 99.9 | 99.7 |
| Claim) | | | | | |
| Total impurities | ≤4.0 | 1.5 | 1.4 | 1.4 | 1.4 |
| Water Content | Report | 3.8 | 4.4 | 4.1 | 4.3 |
| (w/w %) | | | | | |
| Dissolution | Report | Mean, Min, | Mean, Min, | Mean, Min, | Mean, Min, |
| (% Dissolved)^{b} | | Max | Max | Max | Max |
| 15 min | | 97, 91, 100 | 98, 96, 102 | 95, 84, 97 | 99, 96, 100 |
| 30 min | | 98, 93, 101 | 99, 97, 103 | 98, 95, 100 | 100, 97, 101 |
| 45 min | | 98, 93, 101 | 98, 96, 102 | 98, 96, 100 | 100, 97, 101 |
| 60 min | | 98, 93, 100 | 98, 96, 103 | 98, 96, 100 | 100, 98, 101 |

^{a}Tested at 10 months;
^{b}For experimental conditions see Example 3.

TABLE 24
Stability data (Formulation #7: 20% (w/w), 120 mg sotorasib, uncoated) at 30°
C./65% RH. Tablets were packaged into the 30 count 75 cc HDPE (high density polyethylene)
bottles with silica gel as desiccant, heat induction seal and polypropylene child
resistant closure and placed on stability at conditions specified below.

| | Acceptance | Time (Months) | | | |
|---|---|---|---|---|---|
| Test | Criteria | Initial | 1 | 3 | 6 |
| Description | White to off-white | Conforms | Conforms | Conforms | Conforms^{a} |
| | round tablets with no | | | | |
| | obvious physical defects | | | | |
| Assay (% Label | 90.0 to 110.0 | 102.1 | 99.9 | 98.7 | 100.2 |
| Claim) | | | | | |
| Total impurities | ≤4.0 | 1.5 | 1.5 | 1.4 | 1.5 |
| Water Content | Report | 3.8 | 4.5 | 4.1 | 4.4 |
| (w/w %) | | | | | |
| Dissolution | | Mean, Min, | Mean, Min, | Mean, Min, | Mean, Min, |
| (% Dissolved)^{b} | | Max | Max | Max | Max |
| 15 min | Report | 97, 91, 100 | 99, 97, 103 | 95, 93, 97 | 98, 97, 99 |
| 30 min | | 98, 93, 101 | 100, 98, 103 | 97, 96, 99 | 99, 98, 100 |
| 45 min | | 98, 93, 101 | 100, 98, 103 | 98, 96, 99 | 99, 98, 100 |
| 60 min | | 98, 93, 100 | 99, 98, 103 | 97, 96, 100 | 99, 98, 100 |

^{a}Tested at 10 months;
^{b}For experimental conditions see Example 3.

TABLE 25
Stability data (Formulation #7: 20% (w/w), 120 mg sotorasib, uncoated) at 40°
C./75% RH. Tablets were packaged into the 30 count 75cc HDPE (high density polyethylene)
bottles with silica gel as desiccant, heat induction seal and polypropylene child
resistant closure and placed on stability at conditions specified below.

| | | Time (Months) | | | |
|---|---|---|---|---|---|
| Test | Acceptance Criteria | Initial | 1 | 3 | 6 |
| Description | White to off-white | Conforms | Conforms | Conforms | Conforms^{a} |
| | round tablets with no | | | | |
| | obvious physical defects | | | | |
| Assay (% Label | 90.0 to 110.0 | 102.1 | 99.1 | 99.5 | 99.8 |
| Claim) | | | | | |
| Total impurities | ≤4.0 | 1.5 | 1.4 | 1.4 | 1.5 |
| Water Content | Report | 3.8 | 4.3 | 4.0 | 4.5 |
| (w/w %) | | | | | |
| Dissolution | | Mean, Min, | Mean, Min, | Mean, Min, | Mean, Min, |
| (% Dissolved)^{b} | | Max | Max | Max | Max |
| 15 min | Report | 97, 91, 100 | 100, 98, 101 | 96, 92, 97 | 98, 95, 99 |
| 30 min | | 98, 93, 101 | 100, 98, 101 | 97, 95, 99 | 99, 97, 100 |
| 45 min | | 98, 93, 101 | 100, 99, 101 | 98, 93, 99 | 99, 97, 101 |
| 60 min | | 98, 93, 100 | 100, 98, 101 | 97, 94, 98 | 99, 97, 101 |

^{a}Tested at 10 months;
^{b}For experimental conditions see Example 3.

TABLE 26
Stability data (Formulation #6: 20% (w/w), 30 mg sotorasib) at 5°
C. Tablets were packaged into the 15 count 75cc HDPE (high density polyethylene)
bottles with silica gel as desiccant, heat induction seal and polypropylene
child resistant closure and placed on stability at conditions specified below.

| Formulation | | Time (Months) | | | |
|---|---|---|---|---|---|
| | Acceptance Criteria | Initial | 1 | 3 | 6 |
| Description | White to off-white round | Conforms | Conforms | Conforms | Conforms^{a} |
| | tablets with no obvious | | | | |
| | physical defects | | | | |
| Assay (% Label | 90.0 to 110.0 | 100.3 | 98.5 | 98.1 | 97.6 |
| Claim) | | | | | |
| Total impurities | ≤4.0 | 1.4 | 1.5 | 1.4 | 1.5 |
| Water Content | Report | 3.5 | 2.6 | 4.8 | 4.0 |
| (w/w %) | | | | | |
| Dissolution | Report | Mean, Min, | Mean, Min, | Mean, Min, | Mean, Min, |
| (% Dissolved)^{b} | | Max | Max | Max | Max |
| 15 min | | 98, 96, 100 | 98, 96, 99 | 95, 91, 100 | 93, 92, 95 |
| 30 min | | 99, 96, 100 | 98, 96, 99 | 97, 92, 101 | 95, 93, 97 |
| 45 min | | 99, 97, 101 | 99, 96, 102 | 97, 92, 101 | 96, 93, 98 |
| 60 min | | 99, 97, 100 | 99, 96, 100 | 97, 93, 101 | 96, 93, 97 |

^{a}Tested at 10 months;
^{b}For experimental conditions see Example 3.

TABLE 27
Stability data (Formulation #6: 20% (w/w), 30 mg sotorasib) at 30° C./65%
RH. Tablets were packaged into the 15 count 75cc HDPE (high density polyethylene)
bottles with silica gel as desiccant, heat induction seal and polypropylene child
resistant closure and placed on stability at conditions specified below.

| Formulation | | Time (Months) | | | |
|---|---|---|---|---|---|
| | Acceptance Criteria | Initial | 1 | 3 | 6 |
| Description | White to off-white | Conforms | Conforms | Conforms | Conforms |
| | round tablets with no | | | | |
| | obvious physical | | | | |
| | defects | | | | |
| Assay (% Label | 90.0 to 110.0 | 100.3 | 97.9 | 97.7 | 98.5 |
| Claim) | | | | | |
| Total impurities | ≤4.0 | 1.4 | 1.4 | 1.4 | 1.4 |
| Water Content | Report | 3.5 | 2.5 | 4.4 | 4.5 |
| (w/w %) | | | | | |
| Dissolution | Report | Mean, Min, | Mean, Min, | Mean, Min, Max | Mean, Min, |
| (% Dissolved) | | Max | Max | | Max |
| 15 min | | 98, 96, 100 | 96, 93, 99 | 96, 94, 100 | 94, 88, 97 |
| 30 min | | 99, 96, 100 | 97, 95, 99 | 98, 96, 102 | 97, 95, 98 |
| 45 min | | 99, 97, 101 | 97, 94, 100 | 98, 96, 103 | 97, 96, 99 |
| 60 min | | 99, 97, 100 | 97, 94, 100 | 98, 96, 103 | 97, 96, 99 |

TABLE 28
Stability data (Formulation #6: 20% (w/w), 30 mg sotorasib) at 40° C./75%
RH. Tablets were packaged into the 15 count 75cc HDPE (high density polyethylene)
bottles with silica gel as desiccant, heat induction seal and polypropylene child
resistant closure and placed on stability at conditions specified below.

| Formulation | | Time (Months) | | | |
|---|---|---|---|---|---|
| | Acceptance Criteria | Initial | 1 | 3 | 6 |
| Description | White to off-white | Conforms | Conforms | Conforms | Conforms^{a} |
| | round tablets with no | | | | |
| | obvious physical | | | | |
| | defects | | | | |
| Assay (% Label | 90.0 to 110.0 | 100.3 | 98.2 | 98.8 | 98.6 |
| Claim) | | | | | |
| Total impurities | ≤4.0 | 1.4 | 1.4 | 1.4 | 1.4 |
| Water Content | Report | 3.5 | 2.6 | 4.2 | 4.1 |
| (w/w %) | | | | | |
| Dissolution | Report | Mean, Min, | Mean, Min, | Mean, Min, | Mean, Min, |
| (% Dissolved)^{b} | | Max | Max | Max | Max |
| 15 min | | 98, 96, 100 | 98, 97, 102 | 96, 94, 99 | 94, 91, 95 |
| 30 min | | 99, 96, 100 | 99, 97, 102 | 99, 96, 102 | 96, 93, 98 |
| 45 min | | 99, 97, 101 | 99, 97, 102 | 98, 96, 100 | 96, 93, 97 |
| 60 min | | 99, 97, 100 | 99, 97, 102 | 98, 96, 100 | 96, 94, 98 |

^{a}Tested at 10 months;
^{b}For experimental conditions see Example 3.

Stability data indicated that all testing results meet acceptance criteria: Comparable stability results were observed between Formulation #7 and Formulation #8. For Formulation #6, stability data indicated that all testing results met acceptance criteria with no significant trends observed at storage conditions of 5° C. and 25° C./60% RH for 12 months, and 40° C./75% RH for 6 months. Likewise, stability data for Formulations #7 and #8 met acceptance criteria with no significant trends observed at storage conditions after 3 months under 5C, 30° C./65% RH, and 40° C./75% RH storage conditions.

For the Formulation #8 tablets, a comprehensive accelerated stability assessment program (ASAP) study was conducted, the level of degradation was higher with increasing temperature and humidity, but was within the specification limit at the most stressful condition, 60° C./75% RH (4 weeks). Results from the ASAP study showed that Formulation #8 is stable with respect to temperature and slightly sensitive to humidity. Additionally, at the 4 week timepoint, ssNMR was performed on the Formulation #8 tablets stored at the 60° C./75% RH conditions, and the results confirmed no form change.

An ASAP study was also performed for Formulation #1 (1% (w/w), 1 mg sotorasib), Formulation #6 (20% (w/w), 30 mg sotorasib), Formulation #7 (20% (w/w), 120 mg sotorasib), Formulation #8 (20% (w/w), 120 mg sotorasib), Formulation #4 (30% (w/w), 180 sotorasib), and Formulation #5 (40% (w/w), 360 mg sotorasib). The results are shown below in Tables 29-33.

TABLE 29
Stability data (Formulation #1: 1% (w/w), 1 mg sotorasib). Tablets were stored in open glass vials
and exposed to the temperature and humidity conditions specified below with a study end point of 4 weeks.

| | Timepoint | | | | | | | |
|---|---|---|---|---|---|---|---|---|
| | t = 0 | | t = One week | | t = Two weeks | | t = Four weeks | |
| | Purity, % | Assay | Purity, % | Assay | Purity, % | Assay | Purity, % | Assay |
| Storage condition | Area | (w/w %) | Area | (w/w %) | Area | (w/w %) | Area | (w/w %) |
| 50° C./75% RH | 99.3 | 99.1 | 99.2 | 98.7 | 98.5 | 100.2 | 98.94 | 95.7 |
| 60° C./33% RH | | | 99.3 | 101.1 | 98.8 | 99.5 | 98.68 | 97.3 |
| 60° C./75% RH | | | 99.1 | 100.2 | 98.7 | 99.9 | 98.23 | 96.9 |
| 70° C./33% RH | | | 99.2 | 98.3 | 98.9 | 97.4 | 98.55 | 96.3 |
| 70° C./75% RH | | | 98.7 | 95.8 | 98.0 | 93.8 | 97.23 | 96.4 |

TABLE 30
ASAP stability data (Formulation #6: 20% (w/w),
30 mg sotorasib). Tablets were stored in open glass
vials and exposed to the temperature and humidity conditions
specified below with a study end point of 4 weeks.

| Condition | Purity, % Area |
|---|---|
| Unstressed | 98.6 |
| 50° C./11% RH | 98.6 |
| 50° C./33% RH | 98.6 |
| 50° C./75% RH | 98.5 |
| 60° C./33% RH | 98.6 |
| 60° C./751% RH | 98.3 |

TABLE 31
ASAP stability data for Formulations #7 and #8
(20% (w/w), 120 mg sotorasib). Tablets were stored in
open glass vials and exposed to the temperature and humidity
conditions specified below with a study end point of 4 weeks.

| | |
|---|---|
| Condition | Purity, % Area |

120 mg Uncoated Tablets (Formulation #7)

| | |
|---|---|
| Unstressed | 98.5 |
| 60° C./33% RH | 98.5 |
| 60° C./75% RH | 98.3 |

120 mg Coated Tablets (Formulation #8)

| | |
|---|---|
| Unstressed | 99.7 |
| 40° C./75% RH | 99.5 |
| 50° C./33% RH | 99.6 |
| 50° C./75% RH | 99.5 |
| 60° C./33% RH | 99.5 |
| 60° C./75% RH | 99.4 |

TABLE 32
ASAP stability data for Formulation #4 (30% (w/w),
180 mg sotorasib). Tablets were stored in open glass
vials and exposed to the temperature and humidity conditions
specified below with a study end point of 4 weeks.

| | | Assay | | |
|---|---|---|---|---|
| | | (% Label | | |
| Condition | Time Point | Claim) | Total Degradants (% w/w) | |
| Control # 1 | T = 0 | 98.3 | <0.05 | (0.008) |
| Control # 2 | T = 0 | 96.8 | <0.05 | (0.006) |
| Control # 3 | T = 0 | 97.9 | <0.05 | (0.008) |
| 80° C./75% RH | 7 days | 98.0 | 0.19 | |
| | 14 days | 95.9 | 0.27 | |
| 80° C./33% RH | 7 days | 95.3 | <0.05 | (0.03) |

| | | | |
|---|---|---|---|
| | 14 days | 95.8 | 0.07 |
| 70° C./75% RH | 14 days | 97.2 | 0.21 |
| | 21 days | 96.2 | 0.28 |
| 70° C./33% RH | 14 days | 94.4 | 0.06 |
| | 28 days | 97.9 | 0.11 |
| 60° C./75% RH | 14 days | 98.0 | 0.16 |
| | 28 days | 96.5 | 0.24 |
| 50° C./75% RH | 14 days | 97.6 | 0.06 |
| | 21 days | 96.7 | 0.07 |
| | 28 days | 97.6 | 0.08 |

TABLE 33
ASAP stability data for Formulation #5 (40% (w/w),
360 mg sotorasib). Tablets were stored in open glass
vials and exposed to the temperature and humidity conditions
specified below with a study end point of 4 weeks.

| | | Assay | | |
|---|---|---|---|---|
| | Time | (% Label | | |
| Condition | Point | Claim) | Total Degradants (% w/w) | |
| Control # 1 | T = 0 | 98.9 | <0.05 | (0.009) |
| Control # 2 | T = 0 | 98.4 | <0.05 | (0.008) |
| Control # 3 | T = 0 | 98.0 | <0.05 | (0.008) |
| 80° C./75% RH | 7 days | 98.4 | 0.19 | |
| | 14 days | 98.7 | 0.28 | |
| 80° C./33% RH | 7 days | 97.7 | <0.05 | (0.03) |

| | | | |
|---|---|---|---|
| | 14 days | 97.3 | 0.07 |
| 70° C./75% RH | 14 days | 97.9 | 0.19 |
| | 21 days | 97.2 | 0.26 |
| 70° C./33% RH | 14 days | 97.9 | 0.05 |
| | 28 days | 98.0 | 0.10 |
| 60° C./75% RH | 14 days | 97.6 | 0.13 |
| | 28 days | 98.8 | 0.22 |
| 50° C./75% RH | 14 days | 101.4 | 0.05 |

The ASAPprime® software was further utilized to predict shelf life of the expected commercial packaging configurations using the Zone IVb condition (i.e., 30° C./75% RH). This study supports minimum shelf lives in bottles and UX2000 blisters for 2 years with 99% probability and over 3 years with 95% probability, as shown in Table 34. In addition, a comparison of PVC vs. Aclar® UX2000 (i.e., a moisture protective blister) blisters was performed. The PVC blister did not meet the minimum shelf-life requirement. Finally, 120 count 120 cc bottles will be placed on primary stability.

TABLE 34
ASAPprime ® shelf-life predictions of 120 count 120cc bottles
and blisters at 30° C./75% RH storage conditions.

| Package Type | Blister | Blister | Bottle |
|---|---|---|---|
| Material | PVC | Aclar ® | HDPE heat induction |
| | | UX2000 | sealed |
| Volume | N/A | N/A | 120 cc |
| Number of units | 1 | 1 | 120 |
| Predicted shelf-life years | 1.25 | 2.00 | 2.50 |
| (99% probability)^{a} | | | |
| Predicted shelf-life years | 2.42 | 3.50 | 4.50 |
| (95% probability)^{a} | | | |

^{a}Specification limit of <1.0 for a known Impurity was utilized in predictions

### Example 3—Dissolution Studies

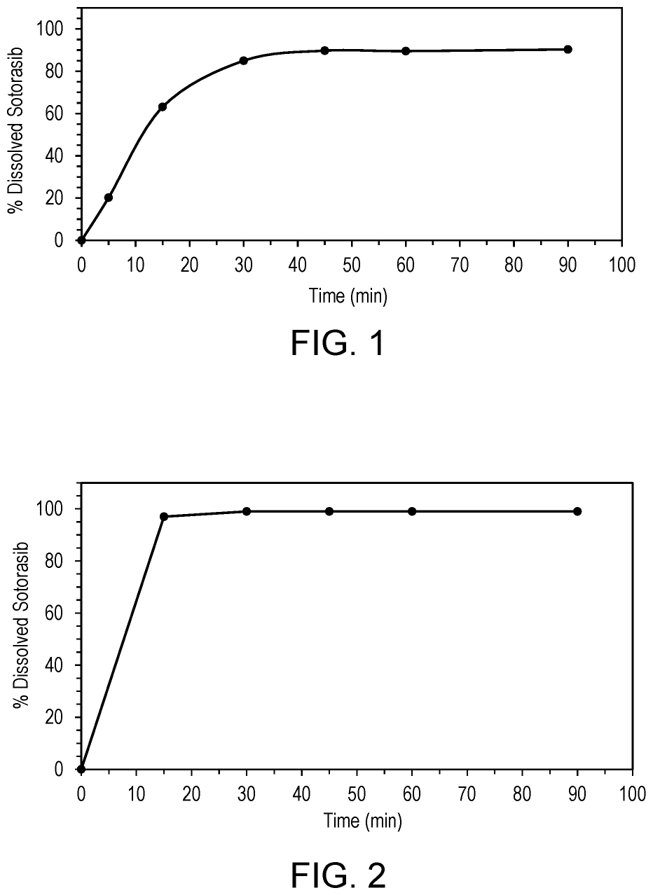

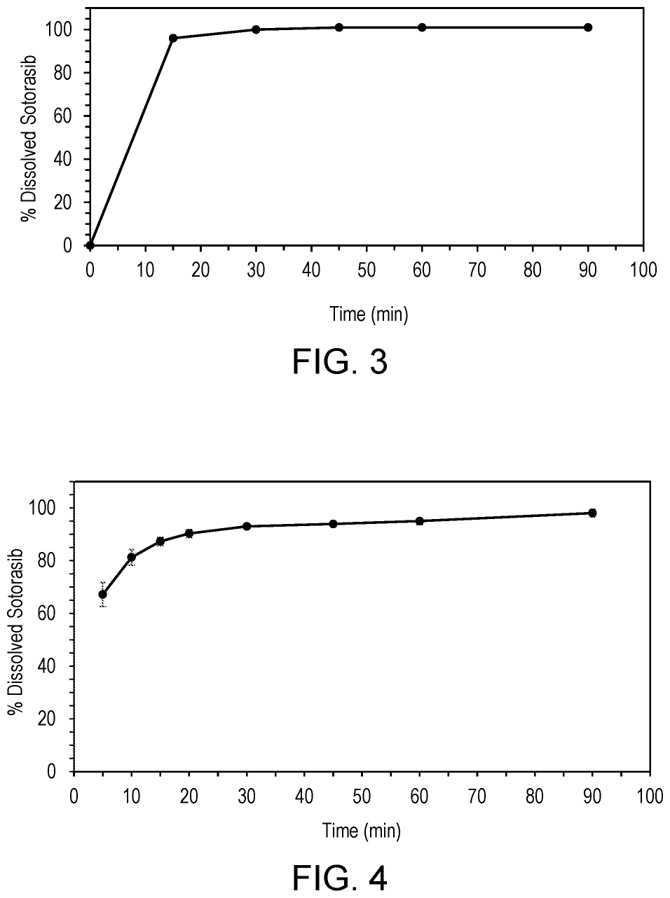

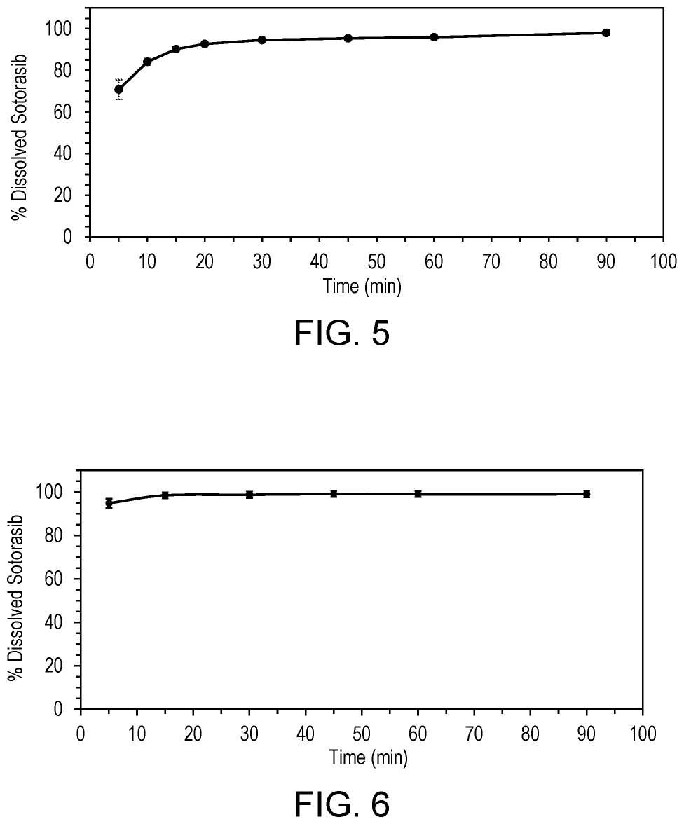

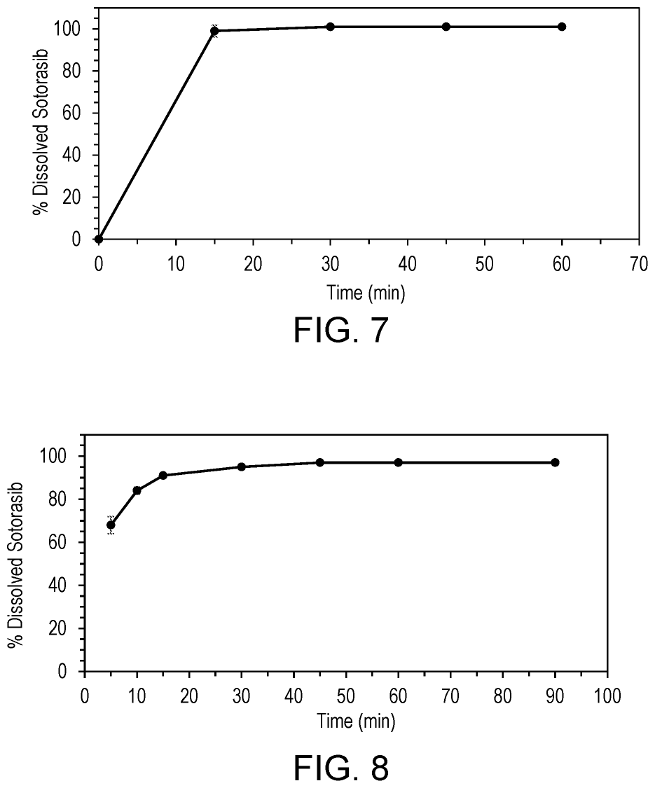

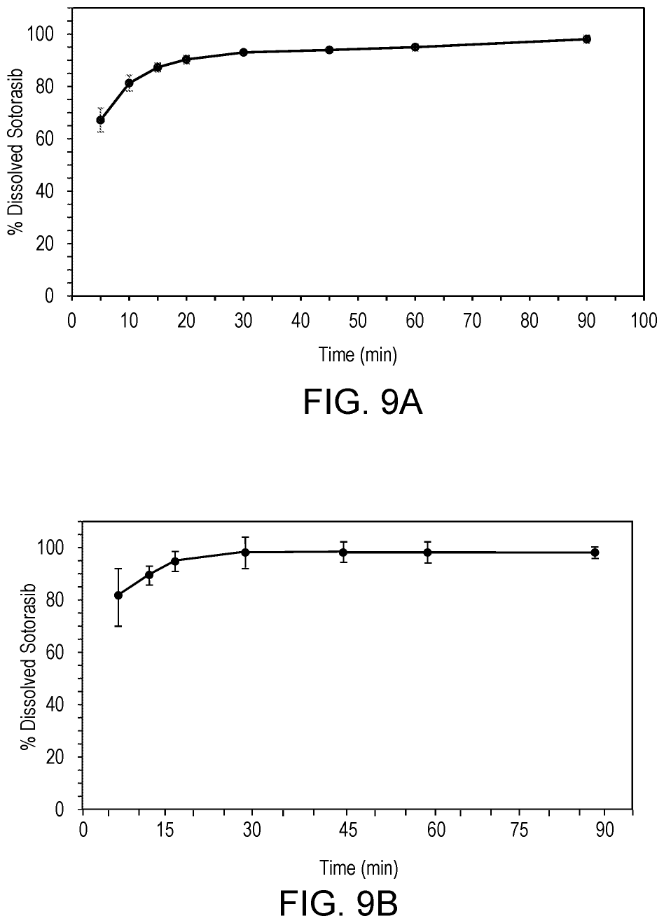

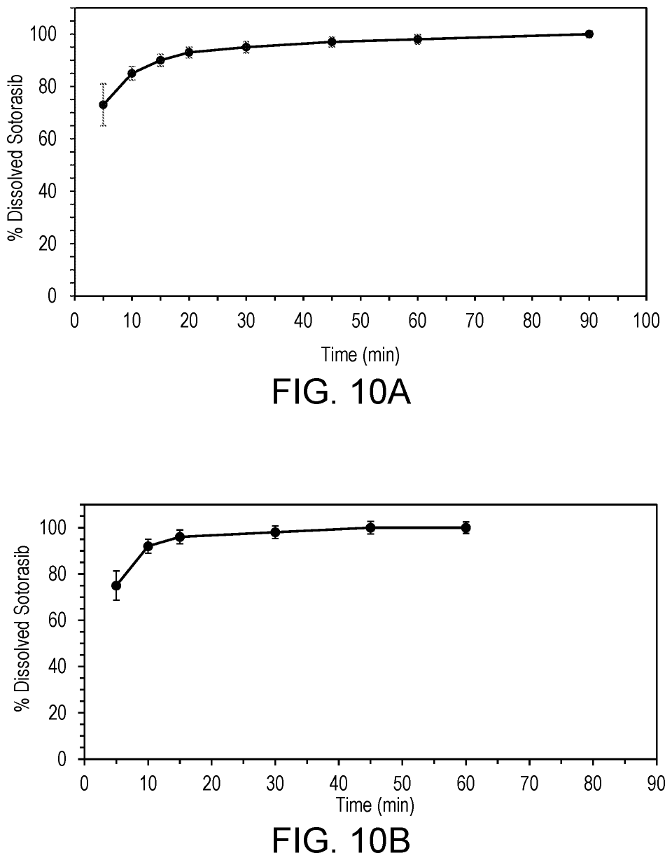

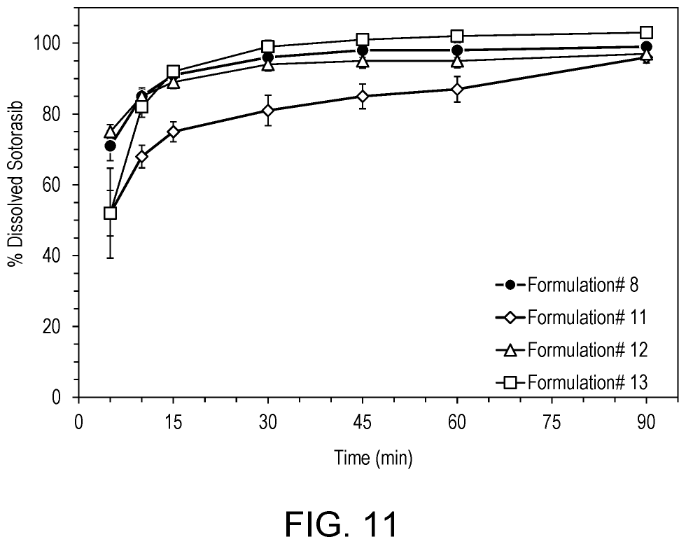

In addition to release and stability data, a comparison of dissolution profiles in multiple media were conducted. See, General Chapter Dissolution <711>, Document ID, 1_GUID-AC788D41-90A2-4F36-A6E7-769954A9ED09_1_en-US, official date May 1, 2016, available at online.uspnf.com (last accessed Apr. 26, 2021). The dissolution medium includes 50 mM sodium phosphate, pH 6.8, appropriate amount of surfactant at 37° C. and 900 mL to achieve sink conditions. The surfactant used in this example was 0.2-1% (w/v) sodium dodecyl sulfate (SDS) for tablets between 1 mg and 360 mg (Table 35). The dissolution method uses an USP<711> apparatus with a 75 rpm paddle speed. See FIGS. 1-11.

TABLE 35
Sodium dodecyl sulfate (SDS) levels used in dissolution media

| | Drug Load | SDS level |
|---|---|---|
| Formulation # | (% w/w, mg) | (% w/v) |
| 1 | 1%, 1 mg | 1.00% |
| 2 | 37.5%, 240 mg | 0.50% |
| 3 | 50%, 360 mg | 0.50% |
| 4 | 30%, 180 mg | 0.30% |
| 5 | 40%, 360 mg | 0.60% |
| 6 | 20%, 30 mg | 0.50% |
| 7 | 20%, 120 mg (uncoated) | 0.50% |
| 8 | 20%, 120 mg (coated) | 0.20% |
| 9a | 32%, 240 mg | 0.40% |
| 9b | 32%, 240 mg | 0.30% |
| 10a | 32%, 320 mg | 0.55% |
| 10b | 32%, 320 mg | 0.40% |
| 11, 12, 13 | 20%, 120 mg | 0.20% |

### Example 4—Water Dispersion Studies

The pharmacokinetics of sotorasib administered as 8×120 mg tablets (Formulation #8) and as tablets pre-dispersed in water was assessed.

Each subject received one administration of sotorasib administered as 8×120 mg tablets (Treatment A) and one administration of sotorasib administered as 8×120 mg tablets dispersed in 240 mL total volume (dose volume+dose container rinses) or water (Treatment B) in either Period 1 or Period 2 according to their assigned group. Doses were administered orally on Days 1 and 4 during the mornings after an overnight fast of at least 10 hours.

A total of 13 subjects entered the study (7 subjects receiving treatment in the sequences of Treatment A followed by Treatment B; and 6 subjects receiving treatment in the sequences of Treatment B followed by Treatment A). Data for all subjects was included in the pharmacokinetic (PK) and safety analyses.

Blood samples were collected for the analysis of plasma concentrations of sotorasib and sotorasib metabolites. The plasma PK concentrations determined for sotorasib and sotorasib metabolite M24 were as follows:
maximum observed plasma concentration (Cₘₐₓ),
area under the plasma concentration-time curve (AUC) from time 0 to the time of last quantifiable concentration (AUCₗₐₛₜ),
AUC from time 0 extrapolated to infinity (AUC_{inf}),
time of Cmax (tₘₐₓ),
apparent terminal elimination half-life (t_{1/2}),
apparent total plasma clearance (CL/F; sotorasib only),
apparent volume of distribution during the terminal phase (Vz/F; sotorasib only),
percentage of AUCinf that is due to extrapolation from the last time of measurable concentration to infinity (% AUCextrap),
elimination rate constant (λ_{z}),
correlation coefficient of terminal elimination phase (R²),
difference between the start and end of exponential fit divided by T_{1/2} (Span ratio),
number of data points included in determination of λ_{z} (Number of points),
lower limit of the terminal phase (start of exponential fit), and
upper limit of terminal phase (end of exponential fit).

Statistical methods: A statistical analysis was conducted to compare sotorasib PK following a tablet dispersed in water (Treatment B) versus that following a sotorasib oral tablet (Treatment A). PK parameters including AUCₗₐₛₜ, AUC_{inf}, and Cₘₐₓ were estimated and compared between Treatment A and Treatment B. The natural log-transformed PK parameters were analyzed using a mixed model. The model included treat, period, and sequence as fixed effect and subject nested within a sequence as a random effect. For each PK parameter separately, (AUCₗₐₛₜ, AUC_{inf}, and Cₘₐₓ), the least squares mean (LSM) for each treatment, difference in LSMs between Treatment A and Treatment B, and corresponding 90% confidence interval (CI) were calculated; these values were then back-transformed to give the geometric last square mean (GSLM), ratio of GLSMs, and corresponding 90% CI. Additionally, the pooled estimate (across all treatments) of the within-subject coefficient of variation was calculated, and residual plots were produced to assess the adequacy of the model(s) fitted.

### Results:

Following administration of sotorasib as tablets dispersed in water (Treatment B), the median sotorasib tₘₐₓ (1 hour) was indicative of rapid absorption and was the same as that observed following administration of sotorasib as tablets (Treatment A). Other sotorasib PK parameters, includes AUCs, Cₘₐₓ and t_{1/2}, were also similar between the two treatments. Sotorasib median time to maximum plasma concentration (tₘₐₓ) and mean half-life (t_{1/2}) were similar when sotorasib was administered as oral tablets to be swallowed and as tablets dispersed in 240 mL of water. Geometric mean sotorasib AUC_{inf} (area under the curve from time zero to infinity) was 25300 h*ng/mL for sotorasib administered as tablets, and 26400 h*ng/mL for sotorasib administered as water dispersion. Geometric mean sotorasib Cₘₐₓ (maximal plasma concentration) was 5440 ng/mL and 5860 ng/mL, respectively.

The ratios (water dispersion/tablet) of the GLSM (90% CI) for sotorasib AUCₗₐₛₜ, AUC_{inf}, and Cₘₐₓ were 1.055 (0.950, 1.171), 1.049 (0.947, 1.162), and 1.080 (0.939, 1.243), respectively, when sotorasib was administered as tablets predispersed in water (Treatment B) and as tablets (Treatment A). The 90% CIs for AUCₗₐₛₜ, AUC_{inf}, and Cₘₐₓ were within 80% to 125% range and spanned unity. Pharmacokinetic parameters for metabolite M24 were also similar between treatments.

Single doses of 960 mg sotorasib as tablets dispersed in water and as tablets were safe and well tolerated when administered to the healthy subjects in the study. There were no serious adverse events, and no treatment-emergent adverse events led to premature discontinuation of a subject from the study. Three treatment-emergent adverse events of constipation, nausea and vomiting were reported during the study and all were considered to be mild and related to sotorasib. All events resolved by the end of the study. There were no clinically significant findings in clinical laboratory evaluations, vital signs, or 12-lead ECGs during the study.

### Conclusion:

In summary, a total dose of 960 mg sotorasib, both as tablets when pre-dispersed in water and as tablets when swallowed as whole tablets were safe and well-tolerated when administered to healthy subjects. Also, when sotorasib was administered as tablets dispersed in water, AUCₗₐₛₜ, AUC_{inf}, and Cₘₐₓ 1.055-, 1.049-, and 1.080-fold of when sotorasib was administered as tablets, respectively, with 90% CIs within the 80% to 125% range.

### Example 5—Mechanical Analysis of Formulation Components

Three dry granulation (roller compaction) placebo blends of microcrystalline cellulose (MCC, Avicel PH102) and lactose (lactose monohydrate, lactose 313), and other individual components including sotorasib, Avicel PH102 and lactose were evaluated for their mechanical properties using the Huxley Bertram (HB) compaction simulator. After compression, tablets were measured for their ejected weight, thickness and diameter. Tablets were then stored for minimally 48 hours to allow for complete viscoelastic relaxation. The recovered dimensions were measured prior to diametrical compression testing performed using the HB compaction simulator operating at a constant loading rate of 5 mm/min. The force required to cause diametrical failure was recorded and used to compute radial tensile strength values.

### Results:

True Density: True density was measured using helium pycnometry. The placebo blend sample (−400-500 mg) was retained after testing due to the non-destructive nature of this measurement.

TABLE 36
True density of sotorasib, placebo blends and individual diluents

| | True Density |
|---|---|
| Material | (g/cm³) (S.D.) |
| Sotorasib | 1.3180 |
| 1:1 MCC:Lactose DG* Blend | 1.5520 (0.0050) |
| 2:1 MCC:Lactose DG* Blend | 1.5553 (0.0002) |
| 3:1 MCC:Lactose DG* Blend | 1.5533 (0.0003) |
| Avicel PH102 | 1.5623 (0.0027) |
| Lactose 313 | 1.5451 (0.0005) |

*DG blend: Dry granulation blend

The true density of the various dry granulated placebo blends agreed favorably with true densities of the primary components, Avicel PH102 and Lactose 313.

Deformation Tendency: During compression, powder particles can deform either reversibly (elastic deformation) or irreversibly (plastic deformation and/or brittle fracture/fragmentation). Pharmaceutical powders are unique in that they almost always exhibit deformation by several different mechanisms with the relative contribution of each varying between materials. The deformation mode that will predominate depends on a number of factors including the compression pressure range of interest, the rate at which the compression pressure is being applied, and the intrinsic mechanical properties of the material. The goal of these studies is to identify the propensity of the formulation blend to deform reversibly, and to distinguish whether its irreversible deformation mechanism is primarily plastic and/or brittle.

Reversible deformation behavior can either be time-independent, or time-dependent. To evaluate both behaviors, a two-stage analysis procedure was used. First, time-independent elastic deformation was quantified by computing the change in solid fraction between the tablet volume at the minimum punch separation distance (in-die) and the tablet volume measured immediately after ejection. Negative values reflect decrease in specimen density. Second, time-dependent elastic deformation, or viscoelastic deformation, was quantified by computing the change in solid fraction between the tablet volume measured immediately after ejection and the tablet volume after storage in ambient conditions for 48 hours.

TABLE 37
Elastic and viscoelastic deformation of
placebo blends (SF = solid fraction)

| | | | ΔSF(%) | ΔSF(%) |
|---|---|---|---|---|
| | ΔSF(%) | ΔSF(%) | (Visco- | (Visco- |
| | (Elastic) | (Elastic) | elastic) | elastic) |
| Material | 50 MPa | 200 MPa | 50 MPa | 200 MPa |
| 1:1 MCC:Lactose DG | −8.80 | −8.61 | −1.32 | −1.04 |
| Blend | | | | |
| 2:1 MCC:Lactose DG | −10.36 | −10.12 | −0.99 | −0.62 |
| Blend | | | | |
| 3:1 MCC:Lactose DG | −10.47 | −10.21 | −1.94 | −1.45 |
| Blend | | | | |

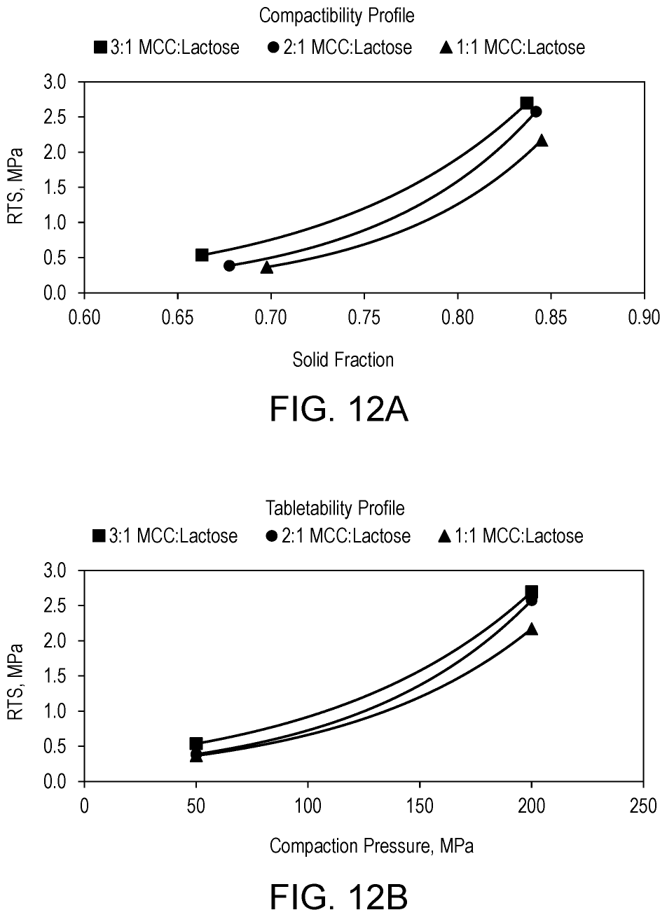

In all cases, the overall extent of reversible deformation is greater for the tablets prepared at 50 MPa than the tablets prepared at 200 MPa. Negative values reflect decreases in specimen density or increases in tablet dimensions. This observation is likely driven by the presence of Avicel PH102 in the formulation. Without wishing to be bound to a particular theory, it is hypothesized that, for a material like Avicel PH102, the internal structure of the compact changes as the pressure increases. This further results in increased amount of energy stored in the compact, which drives reversible deformation. However, the increased tensile strength that is developed at 200 MPa for Avicel PH102 (FIG. 12B) suppresses this reversible deformation, as demonstrated by the less negative values for all placebo blends at 200 MPa compared to 50 MPa. Overall, the data in Table 37 show that all three placebo blends have suitable elastic and viscoelastic deformation properties. Furthermore, this data demonstrates the need for a plastic diluent (e.g., Avicel PH102) to produce an acceptable tablet.

For sotorasib, the values for elastic and viscoelastic recovery were unable to be computed correctly for comparison with the reference material data set. The tablet compressed to 200 MPa experienced lamination upon ejection, which did not allow for proper measurement of the out of die tablet dimensions.

Compressibility: The ability of a powder bed to be reduced in volume due to the application of an applied stress gives an indication of powder compressibility. This behavior is described in terms of tablet solid fraction as a function of compaction pressure (see Table 38). Interpretation of the data considers the change in solid fraction between two pressure conditions. The increased difference in SF at a high pressure and a low pressure is indicative of increased compressibility of the blend. The compressibility of all three placebo blends is on the higher end (due to presence of Avicel PH102) and shows an increasing trend as the amount of Avicel PH102 in the placebo blend increases. Therefore, a 3:1 plastic to brittle diluent ratio, e.g., a 3:1 Avicel PH102 to lactose ratio, is preferred.

TABLE 38
Compressibility of placebo blends (SF = solid fraction)

| | | | Compressibility |
|---|---|---|---|
| | SF at | SF at | (SF_{200 MPa} − |
| Material | 50 MPa | 200 MPa | SF_{50 MPa}) |
| 1:1 MCC:Lactose DG Blend | 0.698 | 0.845 | 0.147 |
| 2:1 MCC:Lactose DG Blend | 0.678 | 0.842 | 0.164 |
| 3:1 MCC:Lactose DG Blend | 0.663 | 0.837 | 0.174 |

For sotorasib, an applied pressure of approximately 145 MPa was required to produce a tablet with an out of die solid fraction of 0.85. In comparison, Avicel PH102 required a pressure of 128 MPa and lactose monohydrate required a pressure of 178 MPa. Accordingly, the presence of additional Avicel PH102 in the formulation should allow reduced compaction pressures to be used to achieve target hardness/tensile strengths. Therefore, a 3:1 plastic to brittle diluent ratio, e.g., a 3:1 Avicel PH102 to lactose ratio, is preferred for a sotorasib formulation.

Compactibility and Tabletability: The ability of a powder bed to cohere into or to form a compact gives an indication of powder compactibility. This behavior is described as a plot of tablet radial tensile strength (RTS) as a function of tablet solid fraction (SF) (FIG. 12A). For understanding fundamental material behavior, it is advantageous to compare materials at similar levels of solid fraction. Higher radial tensile strength at the same solid fraction was observed for the 3:1 MCC:lactose placebo blend (FIG. 12A). The compactibility of all three placebo blends can be classified as “low” because each blend has been previously dry granulated. Tabletability is another relevant parameter which is useful in identifying the pressure needed to achieve a specific tablet hardness or tensile strength. This behavior is described as a plot of tablet radial tensile strength (RTS) as a function of compaction pressure (FIG. 12B). Higher radial tensile strength at a lower compaction pressure was observed for the 3:1 MCC:lactose placebo blend (FIG. 12B). Based upon the compactibility and tabletability profiles, a 3:1 plastic to brittle diluent ratio, e.g., a 3:1 Avicel PH102 to lactose ratio, is preferred for a sotorasib formulation.

TABLE 39
Radial tensile strength (RTS) and compression pressure (CP) of
MCC lactose placebo blends and sotorasib (SF = solid fraction)

| | RTS (MPa) @ | CP (MPa) @ | RTS (MPa) @ | SF @ RTS = |
|---|---|---|---|---|
| Material | 150 MPa | RTS = 2 MPa | 0.85 SF | 2 MPa |
| 1:1 MCC:Lactose DG Blend | 1.57 | 182.0 | N/A | 0.84 |
| 2:1 MCC:Lactose DG Blend | 1.89 | 156.0 | N/A | 0.82 |
| 3:1 MCC:Lactose DG Blend | 2.09 | 143.0 | N/A | 0.80 |
| Sotorasib | 1.62 | 149.00 | 1.59 | 0.85 |

The data in Table 39 shows that as the amount of MCC in the blend increases, the measured radial tensile strength (RTS) at 150 MPa increases as well. Also, the compression pressure (CP) needed to form a tablet having a radial tensile strength of 2 MPa is less for the placebo blends with increasing MCC:lactose ratio. Both trends exhibit non-linear behavior. Overall, the data in Table 39 indicates that a 3:1 plastic to brittle diluent ratio, e.g., a 3:1 Avicel PH102 to lactose ratio, is preferred.

Sotorasib was determined to have a radial tensile strength of 1.62 MPa when compressed to a peak pressure of 150 MPa, and a radial tensile strength of 1.59 MPa when compressed to an out of die solid fraction of 0.85 (see Table 39). The solid fraction at a theoretical strength value of 2 MPa is 0.85 for sotorasib, which indicates high levels of compressibility combined with very low levels of compactibility. The available data suggests that sotorasib is a very weak inter-particulate bond former and, therefore, benefits from a plastic:brittle diluent ratio of 3:1, e.g., a MCC:lactose ratio of 3:1 up to a 20% drug load. For certain formulations at a 20% drug load provided herein, the ratio of plastic diluent (e.g., Avicel PH102) to brittle diluent (e.g., lactose) and sotorasib taken together is 1.46:1 (see Formulations #6, #7, and #8 of Example 1 and Table 40 of Example 6).

The traditional approach to increase the drug load would be, for example, to maintain the ratio of plastic diluent to brittle diluent same and reduce both to accommodate a higher drug load. As discussed previously in Table 39, decreasing the plastic diluent by weight results in lower tensile strength and requires higher compression pressure to produce an acceptable tablet. Because of the lower tensile strength, this decrease in plastic diluent by weight is an inherent liability for higher drug loads made using the traditional approach. Furthermore, the Carr indices of these higher drug load formulations are unfavorable, indicating processability challenges (see Carr index of Formulation 2 and 3 in Table 40 in Example 6). Hence maintaining the ratio of plastic diluent to brittle diluent and sotorasib taken together to 1.4:1 to 1.5:1 is preferred while increasing the drug load. Maintaining this ratio is not feasible using the traditional approach described above.

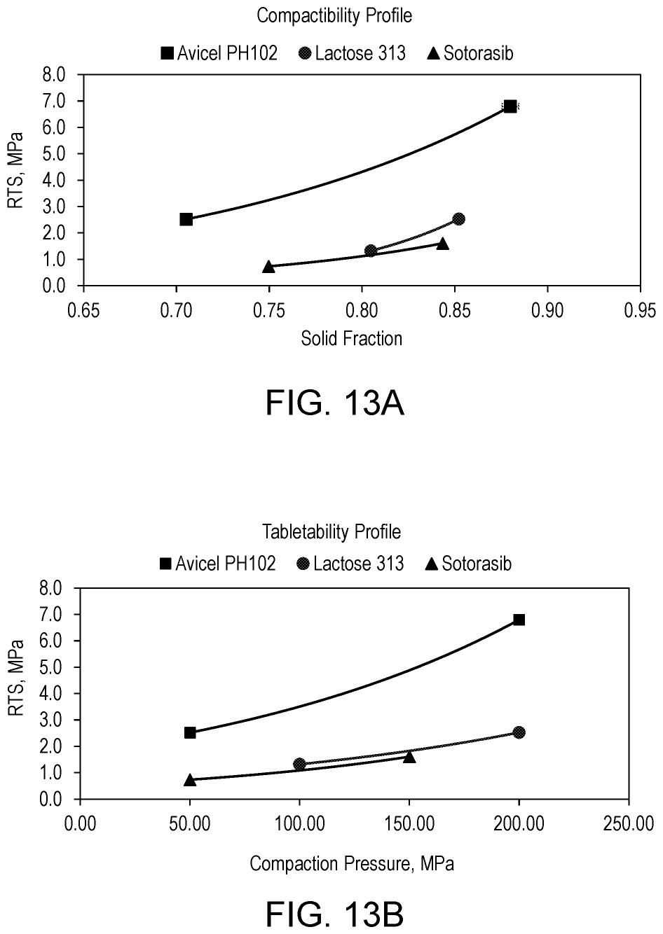

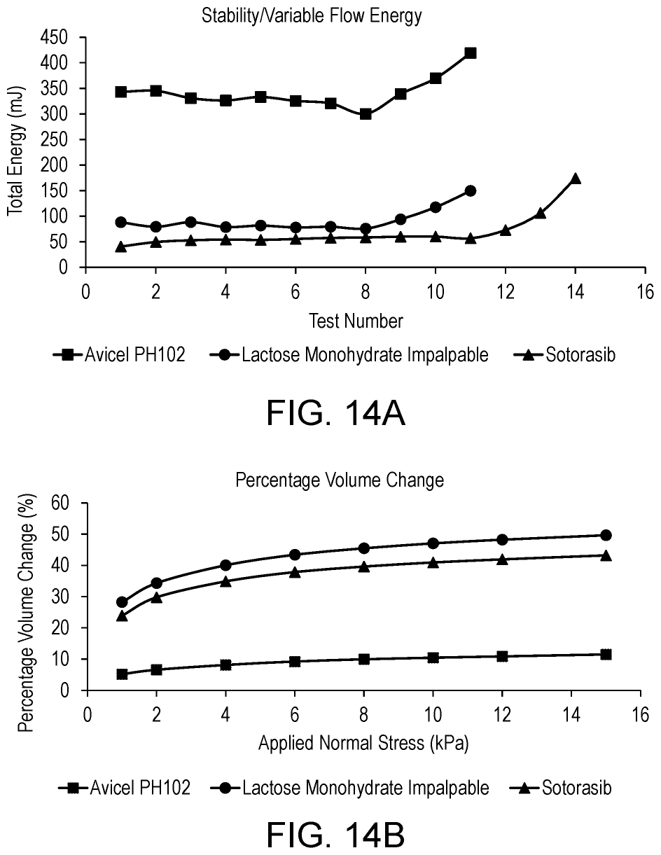

The similarity in mechanical properties between sotorasib and lactose is described in FIG. 13A (compactability) and FIG. 13B (tabletability). The similarity in processability between sotorasib and lactose is described in FIG. 14A (flow energy) and FIG. 14B (percent volume change) of Example 6. These data provide an unexpected and surprising alternate approach to increase the drug load while maintaining the processability (see Formulations #4, #5, #9a, #9b, #10a, and #10b Carr Index in Table 40 of Example 6). The approach involves substituting the brittle diluent, e.g., lactose, with sotorasib, while keeping the ratio of plastic diluent (e.g., MCC) to brittle diluent (e.g., lactose) and sotorasib, taken together, constant between 1.4:1 and 1.5:1 (see Formulations #4, #5, #9a, #9b, #10a and #10b of Example 1).

### Example 6—Flow Energy and Percent Volume Change Studies

This Example describes experiments performed to assess the flow energy and compressibility of some of the formulations described in Example 1.

Flow energy (stability and variable flow) was measured using a powder rheometer. Bulk powder was dispensed into a test cell. Materials were preconditioned with a blade to remove any packing or storage history and to achieve the inherent bulk density of the powder. A blade is passed through the blend at varying speeds to determine the amount of energy required to traverse the powder bed. A stability test is performed at a single blade speed (e.g., 100 mm/sec) with multiple passes. A variable test is performed at a decreasing blade speed (e.g., 100, 70, 40, 10 mm/sec). Stability and variable test data were reported on a single plot showing total energy in mJ versus test number (FIG. 14A).

Percent volume change was measured using a powder rheometer. Bulk powder was dispensed into a test cell. Materials were preconditioned with a blade to remove any packing or storage history and to achieve the inherent bulk density of the powder. A vented piston was inserted into the test cell and applied increasing stress on the powder bed while measuring the volume change. Data are reported on a single plot showing percent volume change vs. applied normal stress in kPa (FIG. 14B).

As shown in FIGS. 14A and 14B, lactose (brittle diluent) and sotorasib have similar properties (i.e., stability/variable flow energy and percent volume change). This data suggests that sotorasib may substitute for brittle components in a formulation, such as a brittle diluent (e.g., lactose). This substitution assists in maintaining certain manufacturability properties, such as flowability of the initial blend of the formulations disclosed herein.

Carr Index: In a free-flowing powder, bulk density and tapped density will be similar in value, therefore, the Carr index will be small. On the other hand, for poor-flowing powder, with more interparticle interaction, the bulk density will be higher than the tapped density, increasing the Carr index. To measure the bulk volume, powder was dispersed into a cylinder. The unsettled apparent or bulk volume (V) of the formulation was recorded. A Tap Density Tester was used to measure the tapped volume. About 10, 500, and 1250 taps on the powder sample were carried out to report the V₁₀, V₅₀₀, and V₁₂₅₀ volumes, respectively. If the difference between V₅₀₀ and V₁₂₅₀ was less than or equal to 1% of the cylinder volume, V₁₂₅₀ was reported as the final tapped volume (V_{f}). If the difference between V₅₀₀ and V₁₂₀ exceeded 1%, increments of 1250 taps were repeated, until the difference between succeeding measurements was less than or equal to 1%. Finally, the Carr index was calculated as follows (results are shown in Table 40):

$$Carr Index = 100 \times \frac{{V}_{o} - {V}_{f}}{{V}_{o}}$$

### Results

Carr index of the initial blend (prior to granulation) was used for relative comparisons of flow between the initial sotorasib formulation blends listed in Table 40.

TABLE 40
Carr indices of sotorasib formulations (initial blend)

| | | | Ratios | | |
|---|---|---|---|---|---|
| | | | | plastic excipient (% | |
| | Drug load and tablet mass | | plastic excipient | w/w):{brittle | |
| | Drug load | Tablet | (% w/w):brittle | excipient (% w/w) + | Carr Index |
| Formulation # | (% w/w, mg) | mass (mg) | excipient (% w/w) | sotorasib (% w/w)} | (Initial blend) |
| 6 | 20, 30 | 150 | 3:1 | 1.46:1 | 40.5 |
| 7, 8 | 20, 120 | 600 | | | |
| 2 | 37.5, 240 | 640 | 3:1 | 0.85:1 | 46.1 |
| 3 | 50, 360 | 720 | 3:1 | 0.56:1 | 49.4 |
| 4 | 30, 180 | 600 | 6.33:1 | 1.46:1 | 40.7 |
| 5 | 40, 360 | 900 | −/− | 1.40:1 | 38.8 |
| 9a, 9b | 32, 240 | 750 | 8.14:1 | 1.46:1 | 43.0 |
| 10a, 10b | 32, 320 | 1000 | 8.14:1 | 1.46:1 | 43.0 |

The data demonstrates that flowability was maintained with 30%, 32% and 40% (w/w) sotorasib high drug load formulations (Formulations #4, #5, #9a, #9b, #10a and #10b) as shown by a Carr index values similar to 20% (w/w) sotorasib formulations (Formulations #6, #7, and #8) by retaining the ratio of plastic diluent (%, w/w) to brittle diluent (% w/w) and sotorasib (% w/w), taken together between 1.4:1 to 1.5:1. In contrast, the high drug load formulations (Formulations #2 and #3), which keep the ratio of plastic to brittle diluent constant (3:1), show a worsening of flowability as expressed by a higher Carr index.

Conclusion—Overall, the data presented in Example 5 and Example 6 demonstrate the benefit of the alternate approach to achieving high drug load formulations (Formulations #4, #5, #9a, #9b, #10a and #10b).

### REFERENCES

Albert et al. 2007 Nat. Methods 4, 903-905.
Alizadeh et al. 1996 Nat. Genet. 14, 457-460.
Amgen Press Release, Dec. 16, 2020; https://wwwext.amgen.com/newsroom/press-releases/2020/12/amgen-submits-sotorasib-new-drug-application-to-u-s-fda-for-advanced-or-metastatic-non-small-cell-lung-cancer-with-kras-g12c-mutation, last accessed Apr. 21, 2021.
Beers and Nederlof 2006 Breast Cancer Res. 8(3), 210.
Bertone et al. 2006 Genome Res 16(2), 271-281.
Canon et al. 2019 Nature 575(7781), 217.
Caunt et al. 2015 Nature Reviews Cancer 15, 577-592.
Cerami et al. 2012 Cancer Discov. 2(5), 401.
Chung et al. 2004 Genome Res. 14(1):188-196.
Cox et al. 2014 J. Nat. Rev. Drug Discov. 13, 828-851.
Dalma-Weiszhausz et al. 2006 Methods Enzymol. 410, 3-28.
Der et al. 1982 Proc. Natl. Acad. Sci. USA. 79, 3637-3640.
Eisenhauer et al. 2009 Eur. J. Cancer 45, 228-247.
Forshew et al. 2012 Sci. Transl. Med. 4, 136ra68.
Gao et al. 2013 Science Signaling 6(269), p 11.
Haber and Velculescu 2014 Cancer Discov. 4, 650-661.
Holderfield et al. 2014 Nat. Rev. Cancer 14, 455-467.
Hong et al. 2020 N. Engl. J. Med. 383, 1207-1217.
Hughes et al. 2001 Nat. Biotechnol. 19(4), 342-347.
Irizarry et al. 2003 Nucleic Acids Res. 31, e15.
Jasmine et al. 2012 PLoS One 7(2), e31968.
Kim et al. 2006 Carcinogenesis 27(3), 392-404.
Kinde et al. 2011 Proc. Natl. Acad. Sci. USA 108, 9530-9535.
Kumar et al. 2012 J. Pharm. Bioallied Sci. 4(1), 21-26.
Laere et al. 2009 Methods Mol. Biol. 512, 71-98.
Lanman et al. 2020 J. Med. Chem. 63, 52-65.
Lin et al. 2010 BMC Genomics 11, 712.
Liu et al. 2017 Biosens Bioelectron 92, 596-601.
Lodes et al. 2009 PLoS One 4(7), e6229.
Malumbres et al. 2003 Nat. Rev. Cancer 3, 459-465.
Mackay et al. 2003 Oncogene 22, 2680-2688.
Mao et al. 2007 Curr. Genomics 8(4), 219-228.
Michels et al. 2007 Genet. Med. 9, 574-584.
Mockler and Ecker 2005 Genomics 85(1), 1-15.
Pinkel et al. 2005 Nat. Genetics 37, S11-S17.
Simanshu et al. 2017 Cell 170, 17-33.
Sridhar et al. 2003 Lancet Oncol. 4, 397-406.
Thomas et al. 2005 Genome Res. 15(12), 1831-1837.
Thompson et al. 2012 PLoS ONE 7, e31597.
Vojtek et al. 1998 J. Biol. Chem., 273, 19925-19928.
Wang et al. 2012 Cancer Genet 205(7-8), 341-355.
Wei et al. 2008 Nucleic Acids Res 36(9), 2926-2938.
Zhang et al. 2017 Materials 10, 845 (pages 1-16).

What is claimed is:

1. A formulation comprising
(a) sotorasib;
(b) a diluent in an amount of 40-95% (w/w),
(c) a disintegrant in an amount of 0.5-5% (w/w), and
(d) a lubricant in an amount of 0.25-5% (w/w).

2. The formulation of claim 1, comprising sotorasib in an amount of 1-50% (w/w).

3. The formulation of claim 1 or claim 2, wherein the diluent comprises one or more of lactose, dibasic calcium phosphate (DCP), mannitol, sorbitol, xylitol, calcium carbonate, magnesium carbonate, tribasic calcium phosphate, trehalose, microcrystalline cellulose, and starch.

4. The formulation of any one of claims 1-3, wherein the diluent comprises one or more of lactose, dibasic calcium phosphate (DCP), mannitol, microcrystalline cellulose, and starch.

5. The formulation of any one of claims 1-4, wherein the diluent comprises one or more of lactose and microcrystalline cellulose.

6. The formulation of any one of claims 1-4, wherein the diluent comprises one or more of lactose and starch.

7. The formulation of any one of claims 1-4, wherein the diluent comprises one or more of lactose, dibasic calcium phosphate (DCP), and mannitol.

8. The formulation of any one of claims 1-4 and 6, wherein starch is pregelatinized starch or corn starch.

9. The formulation of any one of claims 3-7, wherein lactose is lactose monohydrate.

10. The formulation of claim 1, comprising sotorasib in an amount of 1-20% (w/w).

11. The formulation of claim 10, comprising sotorasib in an amount of 20% (w/w).

12. The formulation of claim 10 or claim 11, comprising the diluent in an amount of 61-91% (w/w).

13. The formulation of claim 10 or claim 11, comprising the diluent in an amount of 76% (w/w).

14. The formulation of any one of claims 10-13, wherein the diluent comprises a plastic diluent and a brittle diluent, wherein the ratio by weight of the plastic diluent to the brittle diluent ranges from 2.5:1 to 3.5:1.

15. The formulation of claims 10-13, wherein the diluent comprises a plastic diluent and a brittle diluent, wherein the ratio by weight of the plastic diluent to the brittle diluent is 3:1.

16. The formulation of claim 1, comprising sotorasib in an amount of 20-45% (w/w).

17. The formulation of claim 16, comprising sotorasib in an amount of 20% (w/w).

18. The formulation of claim 16, comprising sotorasib in an amount of 32% (w/w).

19. The formulation of claim 16 or claim 18, comprising the diluent in an amount of 51-77% (w/w)

20. The formulation of claim 16 or claim 18, comprising the diluent in an amount of 64% (w/w).

21. The formulation of any one of claims 16-21, wherein the diluent comprises a plastic diluent and optionally a brittle diluent, wherein the ratio by weight of the plastic diluent to sotorasib and the brittle diluent, if present, taken together, ranges from 1.2:1 to 1.7:1.

22. The formulation of any one of claims 16-21, wherein the diluent comprises a plastic diluent and optionally a brittle diluent, wherein the ratio by weight of the plastic diluent to sotorasib and the brittle diluent, if present, taken together, ranges from 1.4:1 to 1.5:1.

23. The formulation of claim 1, comprising the diluent in an amount of 61-91% (w/w).

24. The formulation of claim 1, comprising the diluent in an amount of 76% (w/w).

25. The formulation of claim 1, comprising the diluent in an amount of 51-77% (w/w).

26. The formulation of claim 1, comprising the diluent in an amount of 64% (w/w).

27. The formulation of any one of claims 23-26, wherein diluent comprises a plastic diluent and optionally a brittle diluent, and wherein
(a) provided that the brittle diluent is present, the formulation is characterized by
(1) a first ratio by weight of the plastic diluent to the brittle diluent that is greater than or equal to 2.5:1, 2.7:1, 3:1, 3.3:1, or 3.5:1; and
(2) a second ratio by weight of the plastic diluent to sotorasib and the brittle diluent, taken together, is greater than or equal to 1.2:1, 1.4:1, 1.5:1, or 1.7:1 and less than the first ratio; or
(b) provided that the brittle diluent is absent, the formulation is characterized by a ratio by weight of the plastic diluent to sotorasib that is greater than or equal to 1.2:1, 1.4:1, 1.5:1, or 1.7:1 and less than 2.5:1, 2.7:1, 3:1, 3.3:1, or 3.5:1.

28. The formulation of claim 27, wherein the diluent comprises a plastic diluent and a brittle diluent, and wherein the first ratio is greater than or equal to 3:1 and the second ratio is greater than or equal to 1.4:1 and less than 3:1.

29. The formulation of any one of claims 23-26, wherein the diluent comprises a plastic diluent and no brittle diluent, and wherein the ratio by weight of the plastic diluent to sotorasib that is greater than or equal to 1.4:1 and less than 3:1.

30. The formulation of any one of claims 14-15, 21, 22, and 27-29, wherein the plastic diluent comprises one or more of microcrystalline cellulose and starch.

31. The formulation of claim 30, wherein the plastic diluent is microcrystalline cellulose.

32. The formulation of claim 30, wherein the plastic diluent is starch.

33. The formulation of claim 30 or claim 32, wherein starch is pregelatinized starch or corn starch.

34. The formulation of any one of claims 14-15, 21, 22, 27, and 28, wherein the brittle diluent comprises one or more of lactose, dibasic calcium phosphate (DCP), mannitol, sorbitol, xylitol, calcium carbonate, magnesium carbonate, tribasic calcium phosphate, and trehalose.

35. The formulation of claim 34, wherein the brittle diluent comprises one or more of lactose, dibasic calcium phosphate (DCP), or mannitol.

36. The formulation of claim 34, wherein the brittle diluent is lactose.

37. The formulation of any one of claims 34-36, wherein the lactose is lactose monohydrate.

38. The formulation of any one of claims 1-37, comprising a disintegrant in an amount of 1-5% (w/w).

39. The formulation of any one of claims 1-37, comprising a disintegrant in an amount of 3% (w/w).

40. The formulation of any one of claims 1 and 38-39, wherein the disintegrant comprises one or more of cross-linked sodium carboxy methyl cellulose (croscarmellose sodium), cross-linked polyvinylpyrrolidone (crospovidone), sodium starch glycolate, pregelatinized starch, calcium carboxymethyl cellulose, low substituted hydroxypropyl cellulose, and magnesium aluminum silicate.

41. The formulation of claim 40, wherein the disintegrant comprises one or more of croscarmellose sodium and sodium starch glycolate.

42. The formulation of claim 40, wherein the disintegrant is croscarmellose sodium.

43. The formulation of any one of claims 1-42, comprising a lubricant in an amount of 0.5-3% (w/w).

44. The formulation of any one of claims 1-42, comprising a lubricant in an amount of 1% (w/w).

45. The formulation of any one of claims 1 and 43-44, wherein the lubricant comprises one or more of magnesium stearate, calcium stearate, oleic acid, caprylic acid, stearic acid, magnesium isovalerate, calcium laurate, magnesium palmitate, behenic acid, glyceryl behenate, glyceryl stearate, sodium stearyl fumarate, potassium stearyl fumarate, zinc stearate, sodium oleate, sodium stearate, sodium benzoate, sodium acetate, sodium chloride, talc, polyethylene glycol, and hydrogenated vegetable oil.

46. The formulation of claim 45, wherein the lubricant is magnesium stearate.

47. The formulation of any one of claims 1-46, comprising sotorasib in an amount of 1 mg to 360 mg.

48. The formulation of any one of claims 1-46, comprising sotorasib in an amount of 120 mg.

49. The formulation of any one of claims 1-46, comprising sotorasib in an amount of 240 mg.

50. The formulation of any one of claims 1-46, comprising sotorasib in an amount of 320 mg.

51. The formulation of any one of claims 1-9, 40-42, 45, and 46, comprising sotorasib in an amount of 16-24% (w/w), a diluent in an amount of 61-91% (w/w), a disintegrant in an amount of 2.4-3.6% (w/w), and a lubricant in an amount of 0.8-1.2% (w/w).

52. The formulation of any one of claims 1-9, 40-42, 45, and 46, comprising sotorasib in an amount of 20% (w/w), a diluent in an amount of 76% (w/w), a disintegrant in an amount of 3% (w/w), and a lubricant in an amount of 1% (w/w).

53. The formulation of any one of claims 51-52, comprising sotorasib in an amount of 120 mg.

54. The formulation of any one of claims 1-9, 40-42, 45, and 46, comprising sotorasib in an amount of 26-38% (w/w), a diluent in an amount of 51-77% (w/w), a disintegrant in an amount of 2.4-3.6% (w/w), and a lubricant in an amount of 0.8-1.2% (w/w).

55. The formulation of any one of claims 1-9, 40-42, 45, and 46, comprising sotorasib in an amount of 32% (w/w), a diluent in an amount of 64% (w/w), a disintegrant in an amount of 3% (w/w), and a lubricant in an amount of 1% (w/w).

56. The formulation of any one of claims 54-55, comprising sotorasib in an amount of 240 mg.

57. The formulation of any one of claims 54-55, comprising sotorasib in an amount of 320 mg.

58. The formulation of any one of claims 1-57, wherein the formulation is a solid dosage form.

59. The formulation of claim 58, wherein the solid dosage form is for oral administration.

60. The formulation of claim 58 or claim 59, wherein the solid dosage form is a tablet.

61. The formulation of claim 60, wherein the tablet is coated with a coating composition.

62. The formulation of claim 64, wherein the coating composition comprises polyvinyl alcohol.

63. The formulation of claim 62, wherein the coating composition further comprises one or more of titanium dioxide, polyethylene glycol, talc, and a coloring agent.

64. The formulation of any one of claim 1-63, wherein at least 50% of the sotorasib in the formulation is released within 30 minutes as measured by a dissolution test using a USP <711> apparatus 2 with 75 rpm paddle speed, at 37° C. in a dissolution medium of 900 ml of water at pH 6.7 comprising 50 mM sodium phosphate and a surfactant to maintain sink conditions.

65. The formulation of claim 64, wherein at least 80% of the sotorasib in the formulation is released within 30 minutes.

66. The formulation of claim 64, wherein at least 85% of the sotorasib in the formulation is released within 15 minutes.

67. The formulation of any one of claims 64-66, wherein the surfactant is 0.2-0.6% (w/v) sodium dodecyl sulfate (SDS).

68. The formulation of any one of claims 64-67, wherein the formulation comprises sotorasib in an amount of 120 mg and the dissolution medium comprises 0.5% (w/v) sodium dodecyl sulfate (SDS).

69. The formulation of any one of claims 64-67, wherein the formulation comprises sotorasib in an amount of 240 mg and the dissolution medium comprises 0.3% (w/v) sodium dodecyl sulfate (SDS).

70. The formulation of any one of claims 64-67, wherein the formulation comprises sotorasib in an amount of 320 mg and the dissolution medium comprises 0.4% (w/v) sodium dodecyl sulfate (SDS).

71. A method of treating cancer in a patient, the method comprising administering to the patient a therapeutically effective amount of sotorasib provided as the formulation of any one of claims 1-66, wherein the formulation provides the therapeutically effective amount in one or more dosage units.

72. The method of claim 71, wherein one or more cells of the cancer express a KRAS G12C mutant protein.

73. The method of claim 71 or claim 72, wherein the therapeutically effective amount is 240 mg.

74. The method of claim 73, wherein the therapeutically effective amount is provided by the formulation of claim 48 or claim 53 in two dosage units.

75. The method of claim 73, wherein the therapeutically effective amount is provided by the formulation of claim 49 or claim 56 in one dosage unit.

76. The method of claim 71 or claim 72, wherein the therapeutically effective amount is 960 mg.

77. The method of claim 76, wherein the therapeutically effective amount is provided by the formulation of claim 48 or claim 53 in eight dosage units.

78. The method of claim 76, wherein the therapeutically effective amount is provided by the formulation of claim 49 or claim 56 in four dosage units.

79. The method of claim 76, wherein the therapeutically effective amount is provided by the formulation of claim 50 or claim 57 in three dosage units.

80. The method of any one of claims 71-79, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

81. The method of any one of claims 71-80, wherein the method further comprises dispersing the therapeutically effective amount provided as one or more dosage units in water by stirring before administration to the patient.

82. The method of claim 81, wherein the water is non-carbonated.

83. The method of claim 81 or claim 82, wherein the water has room-temperature.

84. The method of any one of claims 81-83, wherein the water has a volume of 120 mL.

85. The method of any one of claims 81-84, wherein the therapeutically effective amount is dispersed in water immediately or within 2 hours before administration to the patient.

86. The method of any one of claims 81-85, wherein the patient has difficulty swallowing solids.
